(19) United States
(12) Patent Application Publication
(10) Pub. No.: US 2025/0109170 A1
(43) Pub. Date: Apr. 3, 2025

(54) METHOD FOR MANUFACTURING GANIRELIX

(71) Applicant: ANYGEN CO., LTD., Gwangju (KR)

(72) Inventors: JAE IL KIM, Gwangju (KR); KOOK SANG HWANG, Gyeonggi-do (KR); JU YOUNG LEE, Daejeon (KR); DONG MIN KIM, Sejong (KR); EUN JIN JO, Chungcheongbuk-do (KR); SEUL GI LEE, Chungcheongbuk-do (KR)

(21) Appl. No.: 18/715,313

(22) PCT Filed: Dec. 1, 2022
(86) PCT No.: PCT/KR2022/019416

§ 371 Date: May 31, 2024

(30) Foreign Application Priority Data
Dec. 1, 2021 (KR) 10-2021-0169772
Dec. 1, 2022 (KR) 10-2022-0165580

(51) Int. Cl. C07K 7/23

(52) U.S. Cl. CPC C07K 7/23

(57) ABSTRACT

In a method for manufacturing ganirelix, a peptide intermediate A represented by Chemical Formula 19 is obtained, a peptide intermediate B represented by Chemical Formula 21 is obtained, and ganirelix represented by Chemical Formula 13 is obtained through a convergent synthesis of the peptide intermediate A and the peptide intermediate B. Ganirelix can be obtained in high purity and high yield, and the commercial mass-production process therefor is feasible, and also, an economical advantage, that is, the reduction in the production costs compared to conventional technologies, is provided. Furthermore, it is possible to obtain a large quantity of Ganirelix more safely than conventional technologies.

### CROSS REFERENCE TO RELATED APPLICATIONS AND CLAIM OF PRIORITY

This application claims benefit under 35 U.S.C. 119, 120, 121, or 365 (c), and is a National Stage entry from International Application No. PCT/KR2022/019416, filed Dec. 1, 2022, which claims priority to the benefit of Korean Patent Application Nos. 10-2021-0169772 filed on Dec. 1, 2021, and 10-2022-0165580 filed on Dec. 1, 2022, in the Korean Intellectual Property Office, the entire contents of which are incorporated herein by reference.

### BACKGROUND

### 1. Technical Field

The present invention relates to a method for manufacturing ganirelix.

### 2. Background Art

A ganirelix acetate injection (product name: ORGAOJTRAN) was researched, developed and produced by MSD (known as Merck in the United States and Canada) and launched in China in 2013 with approval of the China Food and Drug Administration, and is used to prevent the luteinizing hormone (LH) peak from being excessively stimulated. Globally, the incidence of infertility has reached 9%. During the controlled ovarian hyperstimulation (COH) of in vitro fertilization and embryo transfer processes, the use of the regulation of a gonadotropin-releasing hormone (GnRH) agonist has already been commonly adopted. The ganirelix acetate injection is a third-generation GnRH antagonist, and can rapidly and reversibly suppress the release of follicle-stimulating hormone (FSH) and LH in vivo by competitive binding to the GnRH receptor in the anterior pituitary gland. The ganirelix acetate injection provides the latest drug option for preventing the LH peak from prematurely appearing in infertile patients undergoing assisted reproductive technology (ART) controlled ovarian hyperstimulation (COH) therapy.

### SUMMARY

The present invention is directed to providing an optimal mass-production synthesis method capable of obtaining ganirelix including a large number of unnatural amino acids with high purity and high yield.

A technical problem to be solved by the present invention is not limited to the aforementioned problem, and other problems that are not mentioned may be clearly understood by a person skilled in the art from the following description.

Unless otherwise indicated herein, the abbreviations used in the designation of amino acids and protecting groups are based on terms recommended by the Commission of Biochemical Nomenclature of the (Biochemistry, 11:1726-1732 (1972); Pure & Appl. Chem., Vol. 56, No. 5, pp. 595-624, 1984).
CTC resin: 2-Chlorotrityl chloride resin
SPPS: Solid Phase Peptide Synthesis
Fmoc: 9-Fluorenyloxycarbonyl
tBu: tert-Butyl
Tyr: Tyrosine
Ser: Serine
D-3-Pal: 3-(3-Pyridyl)-D-Alanine
D-Phe(4-Cl): 4-choro-D-Phenylalanine
D-2-Nal: 3-(2-Naphthyl)-D-Alanine
Ala: Alanine
Pro: Proline
Leu: Leucine
hArg: homoarginine
D-hArg: D-homoarginine
Et: Ethyl
GNA: Ganirelix acetate
GNA C5: Ganirelix acetate C-term 5mer
GNAN5: Ganirelix acetate N-term 5mer
HCl: Hydrochloric acid
DEPBT: (3-(diethoxyphosphoryloxy)-1,2,3-benzotriazin-4(3H)-one)
DIEA: N,N-Diisopropylethylamine
DIC: N,N′-Diisopropylcarbodiimide
Oxyma: Ethyl(hydroxyimino)cyanoacetate
HATU: Bis(dimethylamino)methylene]-1H-1,2,3-triazolo[4,5-b]pyridinium 3-oxid hexafluorophosphate
HCTU: O-(1H-6-Chlorobenzotriazole-1-yl)-1,1,3,3-tetramethyluronium hexafluorophosphate
HOBt: 1-Hydroxybenzotriazole
HOAt: 1-Hydroxy-7-azabenzotriazole
TBTU: 2-(1H-Benzotriazole-1-yl)-1,1,3,3-tetramethylaminium tetrafluoroborate
TCTU: 1-[Bis(dimethylamino)methylen]-5-chlorobenzotriazolium 3-oxide tetrafluoroborate
TOTU: N-[[[(1-Cyano-2-ethoxy-2-oxoethylidene)amino]oxy](dimethylamino)methylene]-N-methyl-methanaminium tetrafluoroborate
TPTU: 2-(2-Pyridon-1-yl)-1,1,3,3-tetramethyluronium tetrafluoroborate
HBTU: N,N,N′,N′-Tetramethyl-O-(1H-benzotriazol-1-yl) uronium hexafluorophosphate
EDC·HCl: 1-Ethyl-3-(3-dimethylaminopropyl) carbodiimide) hydrochloride
TEA: Triethylamine
TFA: Trifluoroacetic acid
TIS: Triisopropylsilane
NH₄OAc: Ammonium acetate
AcOH: Acetic acid
MeOH: Methanol
DMFDMFDMFDMFDMFDMFDCM: Dichloromethane
DMF: N,N-Dimethylformamide
MTBE: Tert-Butyl methyl ether
ACN: Acetonitrile

One aspect of the present invention provides a method for manufacturing ganirelix, the method including: obtaining a peptide intermediate A represented by the following Chemical Formula 19; obtaining a peptide intermediate B represented by the following Chemical Formula 21; and obtaining ganirelix represented by the following Chemical Formula 13 through the convergent synthesis of intermediate A and intermediate B:

Ac-D-2-Nal-D-Phe(4-Cl)-D-3-Pal-Ser-Tyr-OH [Chemical Formula 19]

H₂N-D-hArg(Et)₂-Leu-hArg(Et)₂-Pro-D-Ala-NH₂ [Chemical Formula 21]

Ac-D-2-Nal-D-Phe(4-Cl)-D-3-Pal-Ser-Tyr-D-hArg(Et)₂-Leu-Arg(Et)₂-Pro-D-Ala-NH₂. [Chemical Formula 13]

Another aspect of the present invention provides a method for manufacturing ganirelix acetate, the method including: obtaining a peptide intermediate A represented by the following Chemical Formula 19; obtaining a peptide intermediate B represented by the following Chemical Formula 21; obtaining ganirelix represented by the following Chemical Formula 13 through the convergent synthesis of intermediate A and intermediate B; and obtaining ganirelix acetate represented by the following Chemical Formula 22 by purifying the obtained ganirelix and substituting the purified ganirelix with an acetate:

Ac-D-2-Nal-D-Phe(4-Cl)-D-3-Pal-Ser-Tyr-OH [Chemical Formula 19]

H₂N-D-hArg(Et)₂-Leu-hArg(Et)₂-Pro-D-Ala-NH₂ [Chemical Formula 21]

Ac-D-2-Nal-D-Phe(4-Cl)-D-3-Pal-Ser-Tyr-D-hArg(Et)₂-Leu-Arg(Et)₂-Pro-D-Ala-NH₂ [Chemical Formula 13]

Ac-D-2-Nal-D-Phe(4-Cl)-D-3-Pal-Ser-Tyr-D-hArg(Et)₂-Leu-hArg(Et)₂-Pro-D-Ala-NH₂·2AcOH. [Chemical Formula 22]

In the manufacturing method, the obtaining of intermediate A may include: obtaining a peptide represented by the following Chemical Formula 18; and removing a protecting group and a resin from the obtained peptide represented by Chemical Formula 18:

Ac-D-2-Nal-D-Phe(4-Cl)-D-3-Pal-Ser(R¹)-Tyr(R¹)—O-Resin [Chemical Formula 18]

(in Chemical Formula 18, R¹ is hydrogen or a hydroxyl group-protecting group).

More specific examples of R¹ may be hydrogen, a tert-butyl group (t-Butyl), a triphenylmethyl group, a 2-chlorotriphenylmethyl group, a benzyl group, a phenyl group, an allyl group, a methyl group, a benzyl phospho group, a 2,2-dimethylpropylsulfo (SO3nP) group, a phosphor group, a 2-chlorotrityl (Clt) group, a dimethylaminoethyl (DMAE) group, a propargyl group or a bis-dimethylamino-phosphono (PO(NMe₂)₂) group, and may be a tert-butyl group according to a preferred exemplary embodiment, but are not limited thereto.

In the manufacturing method, the resin may be a 2-chlorotrityl resin, a trityl resin, a 4-methyltrityl resin, a 4-methoxytrityl resin, or a 4-methylbenzhydrylamine (MBHA) resin, and may be a 2-chlorotrityl resin or an MBHA resin according to a preferred exemplary embodiment, but is not limited thereto.

In the manufacturing method, the removing of the protecting group and the resin may be performed under acidic conditions according to a preferred exemplary embodiment, but may be performed under basic, acidic or neutral conditions or by adjusting the degree of the conditions depending on what protecting group and resin are used, and is not limited thereto.

In the manufacturing method, the removing of the protecting group and the resin may include reacting the peptide represented by Chemical Formula 18 with a mixed solution including a combination of items selected from the group consisting of trifluoroacetic acid (TFA), triisopropylsilane (TIS), dichloromethane (DCM), ethylenedioxy diethanethiol (DODT), dimethylsulfide (DMS) and ammonium iodide (NH₄I), and may include reacting the peptide with a mixed solution including trifluoroacetic acid, triisopropylsilane, or a combination thereof according to a preferred exemplary embodiment, but is not limited thereto.

In the manufacturing method, the removing of the protecting group and the resin may include reacting the peptide represented by Chemical Formula 18 with a mixed solution including TFA and TIS at a volume ratio (v/v) of (35 to 45):(1), (35 to 44):(1), (35 to 43):(1), (35 to 42):(1), (35 to 41):(1) or (35 to 40):(1), and may include reacting the peptide with a mixed solution including TFA and TIS at a volume ratio (v/v) of (35 to 40):(1) according to a preferred exemplary embodiment, but is not limited thereto.

In the manufacturing method, the removing of the protecting group and the resin may include reacting the peptide represented by Chemical Formula 18 with a mixed solution including a combination of items selected from the group consisting of trifluoroacetic acid (TFA), triisopropylsilane (TIS), dichloromethane (DCM), ethylenedioxy diethanethiol (DODT), dimethylsulfide (DMS) and ammonium iodide (NH₄I); and mixing diethyl ether (Et₂O), Tert-Butyl methyl ether (MTBE), or a combination thereof with a reaction solution thereof and solidifying the resulting mixture, but is not limited thereto.

In the manufacturing method, before reacting of the peptide with the mixed solution, and then solidifying of the resulting mixture, the method may further include concentrating the reaction solution to 20% to 40%, 20% to 39%, 20% to 38%, 20% to 37%, 20% to 36%, 20% to 35%, 20% to 34%, 20% to 33%, 20% to 32%, 20% to 31%, 20% to 30%, 21% to 30%, 22% to 30%, 23% to 30%, 24% to 30% or 25% to 30%, and may further include concentrating the reaction solution to 25% to 30% according to a preferred exemplary embodiment, but is not limited thereto.

In the manufacturing method, the solidifying of the resulting mixture may be performed under a nitrogen stream, but is not limited thereto.

In the manufacturing method, the solidifying of the resulting mixture may be performed under a temperature condition of 5° C. to 30° C., 5° C. to 29° C., 5° C. to 28° C., 5° C. to 27° C., 5° C. to 26° C., 5° C. to 25° C., 5° C. to 24° C., 5° C. to 23° C., 5° C. to 22° C., 5° C. to 21° C., 5° C. to 20° C., 6° C. to 20° C., 7° C. to 20° C., 8° C. to 20° C., 9° C. to 20° C. or 10° C. to 20° C., and may be performed under a temperature condition of 10° C. to 20° C. according to a preferred exemplary embodiment, but is not limited thereto.

In the manufacturing method, the obtaining of intermediate A may be performed in the presence of an activator including 2,4,6-collidine, 1-hydroxybenzotriazole (HOBt), ethyl(hydroxyimino)cyanoacetate (Oxyma), N,N′-diisopropylcarbodiimide (DIC), 1-ethyl-3-(3-dimethylaminopropyl) carbodiimide) hydrochloride (EDC·HCl), 1-hydroxy-7-azabenzotriazole (HOAt), 3-(diethoxyphosphoryloxy)-1,2,3-benzotriazin-4(3H)-one (DEPBT), bis(dimethylamino)methylene]-1H-1,2,3-triazolo[4,5-b]pyridinium-3-oxide hexafluorophosphate (HATU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethylaminium tetrafluoroborate (TBTU), 1-[bis(dimethylamino)methylen]-5-chlorobenzotriazolium 3-oxide tetrafluoroborate (TCTU), N-[[[(1-cyano-2-ethoxy-2-oxoethylidene)amino]oxy](dimethylamino)methylene]-N-methyl-methanaminium tetrafluoroborate (TOTU), 2-(2-pyridon-1-yl)-1,1,3,3-tetramethyluronium tetrafluoroborate (TPTU), N,N,N′,N′-tetramethyl-O-(1H-benzotriazol-1-yl) uronium hexafluorophosphate (HBTU), or a combination thereof, but is not limited thereto.

In the manufacturing method, in the obtaining of intermediate A, Tyr may be selected and loaded as a first amino acid, but the method is not limited thereto.

In the manufacturing method, the obtaining of intermediate A may include loading the first amino acid in an amount of 1 eq to 10 eq, 1 eq to 9 eq, 1 eq to 8 eq, 1 eq to 7 eq, 1 eq to 6 eq, 1 eq to 5 eq, 1 eq to 4 eq or 2 eq to 4 eq, and may include loading the first amino acid in an amount of 2 eq to 4 eq according to a preferred exemplary embodiment, but is not limited thereto.

In the manufacturing method, in the obtaining of intermediate A, Ser may be selected and coupled as a second amino acid, but the method is not limited thereto.

In the manufacturing method, the obtaining of intermediate A may include coupling the second amino acid in an amount of 1 eq to 10 eq, 1 eq to 9 eq, 1 eq to 8 eq, 1 eq to 7 eq, 1 eq to 6 eq, 1 eq to 5 eq, 1 eq to 4 eq, or 1 to 3 eq, and may include coupling the second amino acid in an amount of 1 eq to 3 eq according to a preferred exemplary embodiment, but is not limited thereto.

In the manufacturing method, the obtaining of intermediate A may include coupling the second amino acid in the presence of the following basic reagent, but is not limited thereto:

2,4,6-collidine, pyridine, imidazole, pyrrolidine, cyclohexylamine, morpholine, piperidine, 4-methoxypyridine, 2-chloropyridine, 4-dimethylaminopyridine, aniline, 4-methoxyaniline, 4-phenylenediamine, ethylamine, diethylamine, triethylamine, N,N-diisopropylethylamine (DIEA), 1,8-diazabicyclo[5.4.0]undec-7-ene (DBU), or a combination thereof.

In the manufacturing method, the obtaining of intermediate A may include coupling the second amino acid in the presence of the basic reagent in an amount of 0.5 eq to 4 eq, 0.5 eq to 3.5 eq, 0.5 eq to 3 eq, 0.5 eq to 2.5 eq, 0.5 eq to 2 eq, or 1 eq to 2 eq, and may include the second amino acid in the presence of the basic reagent in an amount of 1 eq to 2 eq according to a preferred exemplary embodiment, but is not limited thereto.

In the manufacturing method, in the obtaining of intermediate A, D-3-Pal, D-Phe(4-Cl), and D-2-Nal may be selected and coupled as a third amino acid, a fourth amino acid, and a fifth amino acid, respectively, but the method is not limited thereto.

In the manufacturing method, the obtaining of intermediate A may include coupling the third amino acid, the fourth amino acid, and the fifth amino acid in an amount of 1 eq to 10 eq, 1 eq to 9 eq, 1 eq to 8 eq, 1 eq to 7 eq, 1 eq to 6 eq, 1 eq to 5 eq, 1 eq to 4 eq, or 1 eq to 3 eq, and may include coupling the third amino acid, the fourth amino acid, and the fifth amino acid in an amount of 1 eq to 3 eq according to a preferred exemplary embodiment, but is not limited thereto.

In the manufacturing method, the obtaining of intermediate A may be performed under a temperature condition of 5° C. to 40° C., 6° C. to 40° C., 7° C. to 40° C., 8° C. to 40° C., 9° C. to 40° C., 10° C. to 40° C., 10° C. to 39° C., 10° C. to 38° C., 10° C. to 37° C., 10° C. to 36° C., 10° C. to 35° C., 10° C. to 34° C., 10° C. to 33° C., 10° C. to 32° C., 10° C. to 31° C., or 10° C. to 30° C., and may be performed under a temperature condition of 10° C. to 30° C., but is not limited thereto.

In the manufacturing method, the obtaining of intermediate A may be performed under a reaction solvent volume condition of 5 L/mol to 30 L/mol, 5 L/mol to 29 L/mol, 5 L/mol to 28 L/mol, 5 L/mol to 27 L/mol, 5 L/mol to 26 L/mol, or 5 L/mol to 25 L/mol, and may be performed under a reaction solvent volume condition of 5 L/mol to 25 L/mol according to a preferred exemplary embodiment, but is not limited thereto.

In the manufacturing method, the obtaining of intermediate B may include: obtaining a peptide represented by the following Chemical Formula 20; removing a resin from the obtained peptide represented by Chemical Formula 20; and obtaining intermediate B by purifying the peptide from which the resin has been removed, but is not limited thereto:

H₂N-D-hArg(Et)₂-Leu-hArg(Et)₂-Pro-D-Ala-NH-Resin. [Chemical Formula 20]

In the manufacturing method, the resin may be a 2-chlorotrityl resin, a trityl resin, a 4-methyltrityl resin, a 4-methoxytrityl resin, or a 4-methylbenzhydrylamine (MBHA) resin, and may be a 2-chlorotrityl resin or an MBHA resin according to a preferred exemplary embodiment, but is not limited thereto.

In the manufacturing method, the removing of the resin may be performed under acidic conditions according to a preferred exemplary embodiment, but may be performed under basic, acidic or neutral conditions or by adjusting the degree of the conditions depending on what resin is used, and is not limited thereto.

In the manufacturing method, the removing of the resin may include reacting the peptide represented by Chemical Formula 20 with a mixed solution including a combination of items selected from the group consisting of trifluoroacetic acid (TFA), triisopropylsilane (TIS), dichloromethane (DCM), ethylenedioxy diethanethiol (DODT), dimethylsulfide (DMS) and ammonium iodide (NH₄I), and may include reacting the peptide with a mixed solution including trifluoroacetic acid, dichloromethane, or a combination thereof according to a preferred exemplary embodiment, but is not limited thereto.

In the manufacturing method, the removing of the resin may include reacting the peptide represented by Chemical Formula 20 with a mixed solution including TFA and DCM at a volume ratio (v/v) of (0.1 to 5):(1), (0.2 to 5):(1), (0.3 to 5):(1), (0.4 to 5):(1), (0.5 to 5):(1), (0.6 to 5):(1), (0.7 to 5):(1), (0.8 to 5):(1), (0.9 to 5):(1), (1 to 5):(1), (1.1 to 5):(1), (1.2 to 5):(1), (1.3 to 5):(1), (1.4 to 5):(1), (1.5 to 5):(1), (1.5 to 4.5):(1), (1.5 to 4):(1), (1.5 to 3.5):(1), (1.5 to 3):(1), or (2 to 3):(1), and may include reacting the peptide represented by Chemical Formula 20 with a mixed solution including TFA and DCM at a volume ratio (v/v) of (1.5 to 3.5):(1) according to a preferred exemplary embodiment, but is not limited thereto.

In the manufacturing method, the removing of the resin may include reacting the peptide represented by Chemical Formula 20 with a mixed solution including a combination of items selected from the group consisting of trifluoroacetic acid (TFA), triisopropylsilane (TIS), dichloromethane (DCM), ethylenedioxy diethanethiol (DODT), dimethylsulfide (DMS) and ammonium iodide (NH₄I); and mixing diethyl ether (Et₂O), Tert-Butyl methyl ether (MTBE), or a combination thereof with a reaction solution thereof and solidifying the resulting mixture, but is not limited thereto.

In the manufacturing method, the solidifying of the resulting mixture may be performed under a nitrogen stream, but is not limited thereto.

In the manufacturing method, the solidifying of the resulting mixture may be performed under a temperature condition of 5° C. to 30° C., 5° C. to 29° C., 5° C. to 28° C., 5° C. to 27° C., 5° C. to 26° C., 5° C. to 25° C., 5° C. to 24° C., 5° C. to 23° C., 5° C. to 22° C., 5° C. to 21° C., 5° C. to 20° C., 6° C. to 20° C., 7° C. to 20° C., 8° C. to 20° C., 9° C. to 20° C. or 10° C. to 20° C., and may be performed under a temperature condition of 10 to 20° C. according to a preferred exemplary embodiment, but is not limited thereto.

In the manufacturing method, the obtaining of intermediate B may be performed in the presence of an activator including 2,4,6-collidine, 1-hydroxybenzotriazole (HOBt), ethyl(hydroxyimino)cyanoacetate (Oxyma), N,N′-diisopropylcarbodiimide (DIC), 1-ethyl-3-(3-dimethylaminopropyl) carbodiimide) hydrochloride (EDC·HCl), 1-hydroxy-7-azabenzotriazole (HOAt), 3-(diethoxyphosphoryloxy)-1,2,3-benzotriazin-4(3H)-one (DEPBT), bis(dimethylamino)methylene]-1H-1,2,3-triazolo[4,5-b]pyridinium-3-oxide hexafluorophosphate (HATU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethylaminium tetrafluoroborate (TBTU), 1-[bis(dimethylamino)methylen]-5-chlorobenzotriazolium 3-oxide tetrafluoroborate (TCTU), N-[[[(1-cyano-2-ethoxy-2-oxoethylidene)amino]oxy](dimethylamino)methylene]-N-methyl-methanaminium tetrafluoroborate (TOTU), 2-(2-pyridon-1-yl)-1,1,3,3-tetramethyluronium tetrafluoroborate (TPTU), N,N,N′,N′-tetramethyl-O-(1H-benzotriazol-1-yl) uronium hexafluorophosphate (HBTU), or a combination thereof, but is not limited thereto.

In the manufacturing method, the obtaining of intermediate B may include loading or coupling each amino acid in an amount of 1 eq to 5 eq, 1.1 eq to 5 eq, 1.2 eq to 5 eq, 1.3 eq to 5 eq, 1.4 eq to 5 eq, 1.5 eq to 5 eq, 1.6 eq to 5 eq, 1.7 eq to 5 eq, 1.8 eq to 5 eq, 1.9 eq to 5 eq, 2 eq to 5 eq, 2 eq to 4.9 eq, 2 eq to 4.8 eq, 2 eq to 4.7 eq, 2 eq to 4.6 eq, 2 eq to 4.5 eq, 2 eq to 4.4 eq, 2 eq to 4.3 eq, 2 eq to 4.2 eq, 2 eq to 4.1 eq, or 2 eq to 4 eq, and may include loading or coupling each amino acid in an amount of 2 eq to 4 eq according to a preferred exemplary embodiment, but is not limited thereto.

In the manufacturing method, the obtaining of intermediate B may be performed under a reaction solvent volume condition of 5 L/mol to 30 L/mol, 5 L/mol to 29 L/mol, 5 L/mol to 28 L/mol, 5 L/mol to 27 L/mol, 5 L/mol to 26 L/mol, or 5 L/mol to 25 L/mol, and may be performed under a reaction solvent volume condition of 5 L/mol to 25 L/mol according to a preferred exemplary embodiment, but is not limited thereto.

In the manufacturing method, the obtaining of intermediate B may be performed under a temperature condition of 5° C. to 40° C., 6° C. to 40° C., 7° C. to 40° C., 8° C. to 40° C., 9° C. to 40° C., 10° C. to 40° C., 10° C. to 39° C., 10° C. to 38° C., 10° C. to 37° C., 10° C. to 36° C., 10° C. to 35° C., 10° C. to 34° C., 10° C. to 33° C., 10° C. to 32° C., 10° C. to 31° C., or 10° C. to 30° C., and may be performed under a temperature condition of 10° C. to 30° C. according to a preferred exemplary embodiment, but is not limited thereto.

In the manufacturing method, the obtaining of the ganirelix may be performed in the presence of a coupling reagent including 2,4,6-collidine, 1-hydroxybenzotriazole (HOBt), ethyl(hydroxyimino)cyanoacetate (Oxyma), N,N′-diisopropylcarbodiimide (DIC), 1-ethyl-3-(3-dimethylaminopropyl) carbodiimide) hydrochloride (EDC·HCl), 1-hydroxy-7-azabenzotriazole (HOAt), 3-(diethoxyphosphoryloxy)-1,2,3-benzotriazin-4 (3H)-one (DEPBT), bis(dimethylamino)methylene]-1H-1,2,3-triazolo[4,5-b]pyridinium-3-oxide hexafluorophosphate (HATU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethylaminium tetrafluoroborate (TBTU), 1-[bis(dimethylamino)methylen]-5-chlorobenzotriazolium 3-oxide tetrafluoroborate (TCTU), N-[[[(1-cyano-2-ethoxy-2-oxoethylidene)amino]oxy](dimethylamino)methylene]-N-methyl-methanaminium tetrafluoroborate (TOTU), 2-(2-pyridon-1-yl)-1,1,3,3-tetramethyluronium tetrafluoroborate (TPTU), N,N,N′,N′-tetramethyl-O-(1H-benzotriazol-1-yl) uronium hexafluorophosphate (HBTU), or a combination thereof, but is not limited thereto.

In the manufacturing method, the obtaining of the ganirelix may be obtained in the presence of EDC·HCl in an amount of 0.5 eq to 4 eq, 0.5 eq to 3.9 eq, 0.5 eq to 3.8 eq, 0.5 eq to 3.7 eq, 0.5 eq to 3.6 eq, 0.5 eq to 3.5 eq, 0.5 eq to 3.4 eq, 0.5 eq to 3.3 eq, 0.5 eq to 3.2 eq, 0.5 eq to 3.1 eq, 0.5 eq to 3 eq, 0.5 eq to 2.9 eq, 0.5 eq to 2.8 eq, 0.5 eq to 2.7 eq, 0.5 eq to 2.6 eq, 0.5 eq to 2.5 eq, 1 eq to 4 eq, 1 eq to 3.9 eq, 1 eq to 3.8 eq, 1 eq to 3.7 eq, 1 eq to 3.6 eq, 1 eq to 3.5 eq, 1 eq to 3.4 eq, 1 eq to 3.3 eq, 1 eq to 3.2 eq, 1 eq to 3.1 eq, 1 eq to 3 eq, 1 eq to 2.9 eq, 1 eq to 2.8 eq, 1 eq to 2.7 eq, 1 eq to 2.6 eq, or 1 eq to 2.5 eq, and may be performed in the presence of EDC·HCl in an amount of 1 eq to 2.5 eq according to a preferred exemplary embodiment, but is not limited thereto.

In the manufacturing method, the obtaining of the ganirelix may be performed in the presence of HOAt in an amount of 0.2 eq to 3 eq, 0.3 eq to 3 eq, 0.4 eq to 3 eq, 0.5 eq to 3 eq, 0.5 eq to 2.9 eq, 0.5 eq to 2.8 eq, 0.5 eq to 2.7 eq, 0.5 eq to 2.6 eq, 0.5 eq to 2.5 eq, 0.5 eq to 2.4 eq, 0.5 eq to 2.3 eq, 0.5 eq to 2.2 eq, 0.5 eq to 2.1 eq, or 0.5 eq to 2 eq, and may be performed in the presence of HOAt in an amount of 0.5 eq to 2 eq according to a preferred exemplary embodiment, but is not limited thereto.

In the manufacturing method, the obtaining of the ganirelix may include subjecting intermediate A and intermediate B in an amount of 1 eq to 10 eq, 1 eq to 8 eq, 1 eq to 7 eq, 1 eq to 6 eq, 1 eq to 5 eq, 1 eq to 4 eq, 1 eq to 3 eq, or 1 to 2 eq to convergent synthesis, but is not limited thereto.

In the manufacturing method, the obtaining of the ganirelix may include subjecting intermediate A and intermediate B to convergent synthesis in the presence of a coupling reagent in an amount of 1.5 eq to 10 eq, 1.5 eq to 9.5 eq, 1.5 eq to 9 eq, 1.5 eq to 8.5 eq, 1.5 eq to 8 eq, 1.5 eq to 7.5 eq, 1.5 eq to 7 eq, 1.5 eq to 6.5 eq, 1.5 eq to 6 eq, 1.5 eq to 5.5 eq, 1.5 eq to 5 eq, 1.5 eq to 4.5, 1.5 eq to 4 eq, 1.5 eq to 3.5 eq, 1.5 eq to 3 eq, or 1.5 eq to 2.5 eq, and may include subjecting intermediate A and intermediate B to convergent synthesis in the presence of a coupling agent in an amount of 1.5 eq to 2.5 eq according to a preferred exemplary embodiment, but is not limited thereto.

In the manufacturing method, the obtaining of the ganirelix may include: performing a convergent synthesis reaction on intermediate A and intermediate B in the presence of the coupling reagent; and mixing DCM, MTBE, or a combination thereof with a reaction solution thereof and solidifying the resulting mixture, but is not limited thereto.

In the manufacturing method, the solidifying of the resulting mixture may be performed under a nitrogen stream, but is not limited thereto.

In the manufacturing method, in the solidifying of the resulting mixture, DCM and MTBE may be mixed at a volume ratio (v/v) of 1:10 to 10:1, 1:9 to 9:1, 1:8 to 8:1, 1:7 to 7:1, 1:6 to 6:1, 1:5 to 5:1, 1:4 to 4:1, 1:3 to 3:1, or 1:2 to 2:1 in the solution reaction and the resulting mixture may be solidified, and DCM and MTBE may be mixed at a volume ratio (v/v) of 1:2 to 2:1 in the solution reaction and the resulting mixture may be solidified according to a preferred exemplary embodiment, but is not limited thereto.

In the manufacturing method, the solidifying of the resulting mixture may be performed under a temperature condition of 5° C. to 30° C., 5° C. to 2° C., 5° C. to 28° C., 5° C. to 27° C., 5° C. to 26° C., 5° C. to 25° C., 5° C. to 24° C., 5° C. to 23° C., 5° C. to 22° C., 5° C. to 21° C., 5° C. to 20° C., 6° C. to 20° C., 7° C. to 20° C., 8° C. to 20° C., 9° C. to 20° C. or 10° C. to 20° C., and may be performed under a temperature condition of 10° C. to 20° C. according to a preferred exemplary embodiment, but is not limited thereto.

According to the manufacturing method of the present invention, ganirelix can be obtained in high purity and high yield, and the commercial mass-production process therefor is feasible, and also, an economical advantage of reducing production costs compared to conventional technologies is provided. Furthermore, it is possible to obtain a large quantity of ganirelix more safely than with conventional technologies.

However, the effects of the present invention are not limited to the aforementioned effects, and it should be understood to include all possible effects deduced from the configuration of the invention described in the detailed description or the claims.

### BRIEF DESCRIPTION OF THE DRAWINGS

FIG. 1 is a view illustrating a flowchart of the manufacturing process of a linear synthesis method of ganirelix (GNA, TFA form) based on the solid phase synthesis method according to the present invention.

FIG. 2 is a view illustrating a flowchart of the manufacturing process of a convergent synthesis method for ganirelix (GNA, TFA form) with GNA N4 and GNA C6 according to the present invention.

FIG. 3 is a view illustrating a flowchart of the manufacturing process of a convergent synthesis method for ganirelix (GNA, TFA form) with GNA N5 and GNA C5 according to the present invention.

FIG. 4 is a view illustrating a flowchart of the established manufacturing process of a convergent synthesis method for ganirelix acetate (GNA 2AcOH) with GNA N5 and GNA C5 according to the present invention.

FIG. 5 is a view illustrating a soft material change curve according to an increase in DIEA equivalent when coupling a second amino acid (Fmoc-Ser(tBu)-OH) of GNA N5.

FIG. 6 is a view illustrating a purity change curve according to a change in amino acid equivalent from the third amino acid coupling step to the acetylation step of GNA N5.

FIG. 7 is a view illustrating the MALDI-TOF Mass of GNA N5.

FIG. 8 is a view illustrating the MALDI-TOF Mass of GNA C5.

FIG. 9 is a view illustrating the MALDI-TOF Mass of GNA.

### DETAILED DESCRIPTION

Hereinafter, the present invention will be described in detail with reference to examples for more specifically describing the present invention. However, the following examples are provided for illustrative purposes only, and the scope of the present invention is not limited thereto.

### Example 1. Manufacture of Ganirelix by Linear Synthesis

### 1. Swelling

15.9 g of a rink amide MBHA resin (a 4-methylbenzhydrylamine resin: 10 mmol, loading capacity: 0.631 mmol/g) is put into a reactor. 200 mL of DCM is added thereto, the resulting mixture is stirred at 20±5° C. for 30 minutes, and then the solvent is removed.

### 2. Preparation of Fmoc-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20±5° C. for 2 minutes. The washing is repeated five more times. Fmoc-D-Ala-OH (6.3 g, 2.0 eq) and HOBt (3.0 g, 2.2 eq) are dissolved in 100 mL of DMF. This solution is put into the reactor, DIC (2.5 g, 2.0 eq) is added thereto, and then the resulting mixture is stirred at 20±5° C. for 4 hours. After 4 hours of reaction, the reaction solution is removed and the residue is washed with 100 mL of DMF for 2 minutes. The washing is repeated one more time.

### 3. Preparation of Fmoc-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20±5° C. for 2 minutes. The washing is repeated five more times. Fmoc-Pro-OH (6.8 g, 2.0 eq) and HOBt (3.0 g, 2.2 eq) are dissolved in 100 mL of DMF. This solution is put into the reactor, DIC (2.5 g, 2.0 eq) is added thereto, and then the resulting mixture is stirred at 20±5° C. for 4 hours. After 4 hours of reaction, the reaction solution is removed and the residue is washed with 100 mL of DMF for 2 minutes. The washing is repeated one more time.

### 4. Preparation of Fmoc-hArg(Et)₂-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20±5° C. for 2 minutes. The washing is repeated five more times. Fmoc-hArg(Et)₂—OH (10.1 g, 2.0 eq) and HOBt (3.0 g, 2.2 eq) are dissolved in 100 mL of DMF. This solution is put into the reactor, DIC (2.5 g, 2.0 eq) is added thereto, and then the resulting mixture is stirred at 20±5° C. for 4 hours. After 4 hours of reaction, the reaction solution is removed and the residue is washed with 100 mL of DMF for 2 minutes. The washing is repeated one more time.

### 5. Preparation of Fmoc-Leu-hArg(Et)₂-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20±5° C. for 2 minutes. The washing is repeated five more times. Fmoc-Leu-OH (7.1 g, 2.0 eq) and HOBt (3.0 g, 2.2 eq) are dissolved in 100 ml of DMF. This solution is put into the reactor, DIC (2.5 g, 2.0 eq) is added thereto, and then the resulting mixture is stirred at 20±5° C. for 4 hours. After 4 hours of reaction, the reaction solution is removed and the residue is washed with 100 mL of DMF for 2 minutes. The washing is repeated one more time.

### 6. Preparation of Fmoc-D-hArg(Et)₂-Leu-hArg(Et)₂-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20±5° C. for 2 minutes. The washing is repeated five more times. Fmoc-D-hArg(Et)₂—OH (10.1 g, 2.0 eq) and HOBt (3.0 g, 2.2 eq) are dissolved in 100 mL of DMF. This solution is put into the reactor, DIC (2.5 g, 2.0 eq) is added thereto, and then the resulting mixture is stirred at 20±5° C. for 4 hours. After 4 hours of reaction, the reaction solution is removed and the residue is washed with 100 mL of DMF for 2 minutes. The washing is repeated one more time.

### 7. Preparation of Fmoc-Tyr (tBu)-D-hArg(Et)₂-Leu-hArg(Et)₂-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20±5° C. for 2 minutes. The washing is repeated five more times. Fmoc-Tyr (tBu)-OH (9.3 g, 2.0 eq) and HOBt (3.0 g, 2.2 eq) are dissolved in 100 mL of DMF. This solution is put into the reactor, DIC (2.5 g, 2.0 eq) is added thereto, and then the resulting mixture is stirred at 20±5° C. for 4 hours. After 4 hours of reaction, the reaction solution is removed and the residue is washed with 100 mL of DMF for 2 minutes. The washing is repeated one more time.

### 8. Preparation of Fmoc-Ser(tBu)-Tyr (tBu)-D-hArg(Et)₂-Leu-hArg(Et)₂-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20±5° C. for 2 minutes. The washing is repeated five more times. Fmoc-Ser(tBu)-OH (7.7 g, 2.0 eq) and HOBt (3.0 g, 2.2 eq) are dissolved in 100 mL of DMF. This solution is put into the reactor, DIC (2.5 g, 2.0 eq) is added thereto, and then the resulting mixture is stirred at 20±5° C. for 4 hours. After 4 hours of reaction, the reaction solution is removed and the residue is washed with 100 ml of DMF for 2 minutes. The washing is repeated one more time.

### 9. Preparation of Fmoc-D-3-Pal-Ser(tBu)-Tyr (tBu)-D-hArg(Et)₂-Leu-hArg(Et)₂-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20±5° C. for 2 minutes. The washing is repeated five more times. Fmoc-D-3-Pal-OH (7.8 g, 2.0 eq) and HOBt (3.0 g, 2.2 eq) are dissolved in 100 mL of DMF. This solution is put into the reactor, DIC (2.5 g, 2.0 eq) is added thereto, and then the resulting mixture is stirred at 20±5° C. for 4 hours. After 4 hours of reaction, the reaction solution is removed and the residue is washed with 100 mL of DMF for 2 minutes. The washing is repeated one more time.

### 10. Preparation of Fmoc-D-Phe(4-Cl)-D-3-Pal-Ser(tBu)-Tyr (tBu)-D-hArg(Et)₂-Leu-hArg(Et)₂-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20±5° C. for 2 minutes. The washing is repeated five more times. Fmoc-D-Phe(4-Cl)—OH (8.5 g, 2.0 eq) and HOBt (3.0 g, 2.2 eq) are dissolved in 100 mL of DMF. This solution is put into the reactor, DIC (2.5 g, 2.0 eq) is added thereto, and then the resulting mixture is stirred at 20±5° C. for 4 hours. After 4 hours of reaction, the reaction solution is removed and the residue is washed with 100 mL of DMF for 2 minutes. The washing is repeated one more time.

### 11. Preparation of Fmoc-D-2-Nal-D-Phe(4-Cl)-D-3-Pal-Ser(tBu)-Tyr (tBu)-D-hArg(Et)₂-Leu-hArg(Et)₂-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20±5° C. for 2 minutes. The washing is repeated five more times. Fmoc-D-2-Nal-OH (13.2 g, 3.0 eq) and HOBt (4.5 g, 3.3 eq) are dissolved in 150 mL of DMF. This solution is put into the reactor, DIC (4.5 g, 3.0 eq) is added thereto, and then the resulting mixture is stirred at 20±5° C. for 4 hours. After 4 hours of reaction, the reaction solution is removed and the residue is washed with 150 ml of DMF for 2 minutes. The washing is repeated one more time.

### 12. Preparation of Ac-D-2-Nal-D-Phe(4-Cl)-D-3-Pal-Ser(tBu)-Tyr (tBu)-D-hArg(Et)₂-Leu-hArg(Et)₂-Pro-D-Ala-NH-Resin

To remove Fmoc, 150 mL of 20% piperidine in DMF is added to the reactor and the resulting mixture is stirred at 20±5° C. for 15 minutes. The procedure is repeated one more time. The reaction solution is removed and a resin is washed with 150 mL of DMF at 20±5° C. for 2 minutes. The washing is repeated five more times. DIEA (3.9 g, 3.0 eq) is dissolved in 150 mL of DMF and put into the reactor, acetic anhydride (3.1 g, 3.0 eq) is added thereto, and then the resulting mixture is stirred at 20±5° C. for 3 hours. The reaction solution is removed and the residue is washed with DMF (150 mL) at 20±5° C. for 2 minutes. The washing is repeated one more time. The residue is washed with DCM (150 mL) at 20±5° C. for 2 minutes. The washing is repeated one more time. After the resin is dried under vacuum, a peptide represented by the following Chemical Formula 1 (or Chemical Formula 12) is obtained (Chemical Formula 12: Ac-D-2-Nal-D-Phe(4-Cl)-D-3-Pal-Ser (R¹)-Tyr (R¹)-D-hArg(Et)₂-Leu-hArg(Et)₂-Pro-D-Ala-NH-Resin).

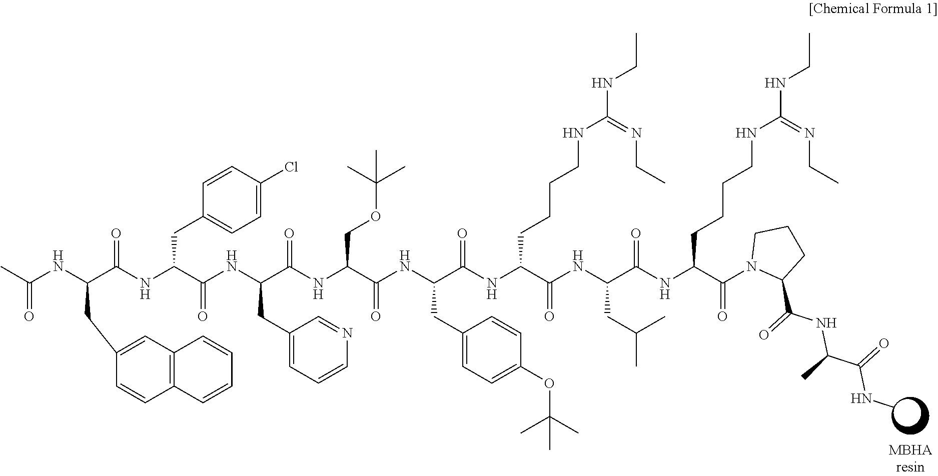 [SMILES: CC/N=C(/NCC)NCCCC[C@H](NC(=O)[C@H](CC(C)C)NC(=O)[C@@H](CCCCN/C(=N\CC)NCC)NC(=O)[C@H](Cc1ccc(OC(C)(C)C)cc1)NC(=O)[C@H](COC(C)(C)C)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N1CCC[C@H]1C(=O)N[C@H](C)C(=O)NC]

### 13. Global Cleavage

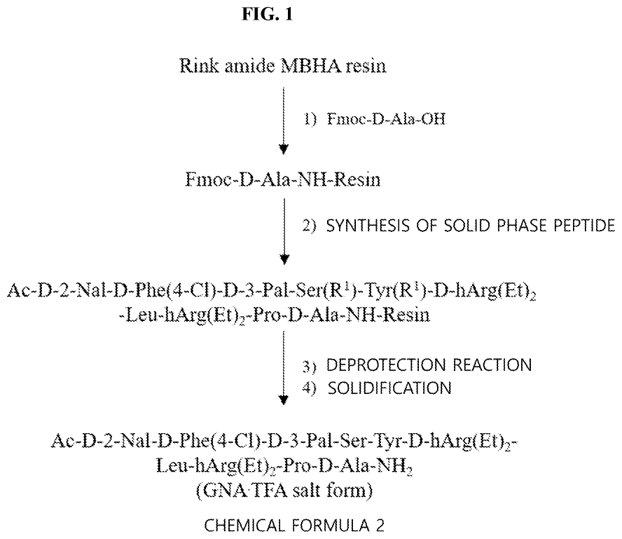

350 mL of a cooled cleavage solution (TFA:TIS:H₂O=95%:2.5%:2.5%, v/v) is slowly added to the dried resin, and the resulting mixture is stirred at 15 to 30° C. for 5 hours. After 3 hours of global cleavage reaction, the completion of the reaction is confirmed through HPLC analysis, and then the cleavage solution is drained and collected. The collected cleavage solution is slowly added dropwise to cooled diethyl ether (cleavage solution:diethyl ether=1:5 (300 mL:1500 mL)) and the resulting mixture is solidified and stirred for 30 minutes. The suspension is filtered under reduced pressure, washed with 1500 mL of MTBE (500 mL×3 times), and then dried. 14.15 g of GNA (TFA salt form, molecular weight: 1570.35, purity: 46.7%, yield: 90.1%) of Chemical Formula 2 (or Chemical Formula 13) was obtained (Chemical Formula 13: Ac-D-2-Nal-D-Phe(4-Cl)-D-3-Pal-Ser-Tyr-D-hArg(Et)₂-Leu-hArg(Et)₂-Pro-D-Ala-NH₂). A flowchart of the entire process is shown in FIG. 1.

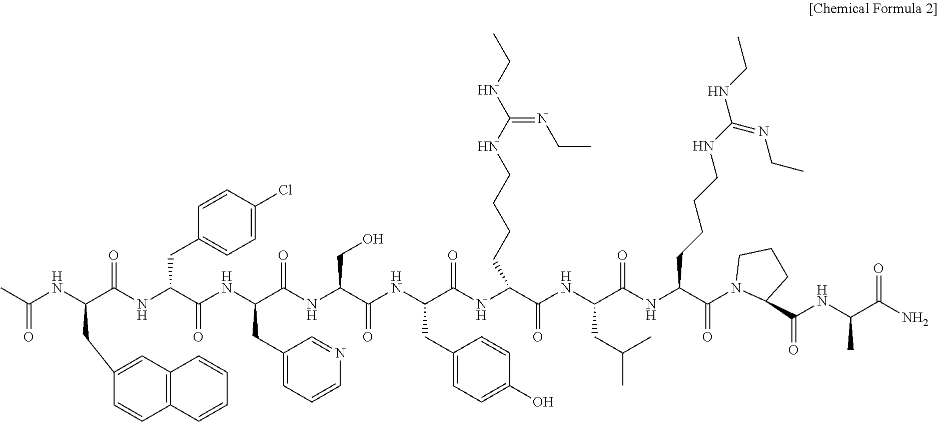 [SMILES: CC/N=C(/NCC)NCCCC[C@H](NC(=O)[C@H](CC(C)C)NC(=O)[C@@H](CCCCN/C(=N\CC)NCC)NC(=O)[C@H](Cc1ccc(O)cc1)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N1CCC[C@H]1C(=O)N[C@H](C)C(N)=O]

### Example 2. Manufacture of Ganirelix by [4+6] Convergent Synthesis

### 1. Synthesis of GNA N4

### (1) Swelling

85.7 g of a 2-chlorotrityl chloride resin (120 mmol, loading capacity: 1.4 mmol/g) is put into a reactor. 1.2 L (10 L/mol) of DCM is added thereto, the resulting mixture is stirred at 20 to 30° C. for 30 minutes, and then the solvent is removed.

### (2) Preparation of Fmoc-Ser(tBu)-O-Resin

Fmoc-Ser(tBu)-OH (92.0 g, 2.0 eq) and DIEA (62.0 g, 4.0 eq) are dissolved in 1.2 L of DCM. This solution is put into the reactor, DIC (31.6 g, 2.5 eq) is added thereto, and then the resulting mixture is stirred at 20±5° C. for 4 hours. After this solution is added to the reactor, the resulting mixture is stirred at 20 to 30° C. for 4 hours. The reaction solution is removed and the residue is washed with 1.0 L of DMF at 20 to 30° C. for 2 minutes. The washing is repeated one more time. 1.0 L of a capping solution (DCM:MeOH:DIEA=85:10:5, v/v) is added to the reactor and the resulting mixture is stirred at 20 to 30° C. for 10 minutes. The capping is repeated one more time. The reaction solution is removed and a resin is washed with 1.0 L of DMF at 20 to 30° C. for 2 minutes. The washing is repeated one more time.

### (3) Preparation of Fmoc-D-3-Pal-Ser(tBu)-O-Resin

To remove Fmoc, 20% piperidine in DMF is added to the reactor and the resulting mixture is stirred at 20±5° C. for 10 minutes. The procedure is repeated one more time. The reaction solution is removed and a resin is washed with 1.1 L of DMF at 20±5° C. for 2 minutes. The washing is repeated two more times. The resin is washed with 1.1 L of DCM at 20 to 30° C. for 2 minutes. The washing is repeated two more times. Fmoc-D-3-Pal-OH (87.8 g, 2.0 eq), HOBt (30.5 g, 2.0 eq), and DIEA (37.6 g, eq) are dissolved in 1.1 L of DMF. This solution is put into the reactor, DIC (31.0 g, 2.0 eq) is added thereto, and then the resulting mixture is stirred at 20±5° C. for 4 hours. The reaction solution is removed and the residue is washed with 1.1 L of DMF for 2 minutes. The washing is repeated one more time.

### (4) Preparation of Fmoc-D-Phe(4-Cl)-D-3-Pal-Ser(tBu)-O-Resin

To remove Fmoc, 20% piperidine in DMF is added to the reactor and the resulting mixture is stirred at 20±5° C. for 10 minutes. The procedure is repeated one more time. The reaction solution is removed and a resin is washed with 1.1 L of DMF at 20±5° C. for 2 minutes. The washing is repeated two more times. The resin is washed with 1.1 L of DCM at 20 to 30° C. for 2 minutes. The washing is repeated two more times. Fmoc-D-Phe(4-Cl)—OH (95.3 g, 2.0 eq) and HOBt (30.5 g, 2.0 eq) are dissolved in 1.1 L of DMF. This solution is put into the reactor, DIC (31.0 g, 2.0 eq) is added thereto, and then the resulting mixture is stirred at 20±5° C. for 4 hours. The reaction solution is removed and the residue is washed with 1.1 L of DMF for 2 minutes. The washing is repeated one more time.

### (5) Preparation of Fmoc-D-2-Nal-D-Phe(4-Cl)-D-3-Pal-Ser(tBu)-O-Resin

To remove Fmoc, 20% piperidine in DMF is added to the reactor and the resulting mixture is stirred at 20±5° C. for 10 minutes. The procedure is repeated one more time. The reaction solution is removed and a resin is washed with 1.1 L of DMF at 20±5° C. for 2 minutes. The washing is repeated two more times. The resin is washed with 1.1 L of DCM at 20 to 30° C. for 2 minutes. The washing is repeated two more times. Fmoc-D-2-Nal-OH (148.3 g, 3.0 eq) and HOBt (45.8 g, 3.0 eq) are dissolved in 1.1 L of DMF 1.1 L. This solution is put into the reactor, DIC (46.4 g, 3.0 eq) is added thereto, and then the resulting mixture is stirred at 20±5° C. for 4 hours. The reaction solution is removed and the residue is washed with 1.1 L of DMF for 2 minutes. The washing is repeated one more time.

### (6) Preparation of Ac-D-2-Nal-D-Phe(4-Cl)-D-3-Pal-Ser(tBu)-O-Resin

To remove Fmoc, 20% piperidine in DMF is added to the reactor and the resulting mixture is stirred at 20±5° C. for 10 minutes. The procedure is repeated one more time. The reaction solution is removed and a resin is washed with 1.1 L of DMF at 20±5° C. for 2 minutes. The washing is repeated two more times. The resin is washed with 1.1 L of DCM at 20 to 30° C. for 2 minutes. The washing is repeated two more times. Acetic anhydride (35.2 g, 3.0 eq) is dissolved in 1.1 mL of DMF and put into the reactor, DIEA (43.6 g, 3.0 eq) is added thereto, and then the resulting mixture is stirred at 20±5° C. for 3 hours. The reaction solution is removed and the residue is washed with 1.1 L of DMF for 2 minutes. The washing is repeated one more time. The resin is washed with 1.1 L of DCM for 2 minutes. The washing is repeated two more times. The resin is dried under vacuum to obtain a peptide represented by the following Chemical Formula 3 (or Chemical Formula 14) (Chemical Formula 14: Ac-D-2-Nal-D-Phe(4-Cl)-D-3-Pal-Ser (R¹)—O-resin

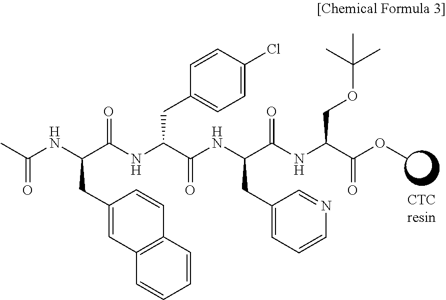 [SMILES: COC(=O)[C@H](COC(C)(C)C)NC(=O)[C@@H](Cc1cccnc1)NC(=O)[C@@H](Cc1ccc(Cl)cc1)NC(=O)[C@@H](Cc1ccc2ccccc2c1)NC(C)=O]

### (7) Global Cleavage Reaction for Preparation of GNA N4 (TFA Salt)

3.0 L of a cooled cleavage solution (TFA:DCM:H₂O=70:29:1, v/v) is slowly added to the dried resin, and the resulting mixture is stirred at 15 to 30° C. for 3 hours. After 3 hours of global cleavage reaction, the completion of the reaction is confirmed through HPLC analysis, and then the cleavage solution is drained and collected. The reaction solution is solidified by adding dropwise 15 L of cooled diethyl ether (cleavage reaction solution:diethyl ether=1:5), and stirred for 30 minutes. The suspension was filtered under reduced pressure, washed with diethyl ether (5.0 L×3 times), and dried under nitrogen vacuum for 15 hours or more to obtain 76.5 g of GNA N4 of Chemical Formula 4 (TFA salt form, molecular weight: 674.15 g/mol, purity: 95.5%, and yield: 94.6%) (Chemical Formula 15: Ac-D-2-Nal-D-Phe(4-Cl)-D-3-Pal-Ser-OH).

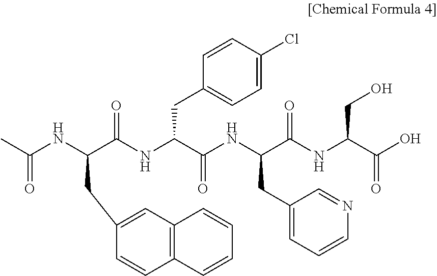 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)O]

### 2. GNA C6

### (1) Swelling

31.3 g of a rink amide MBHA resin (20 mmol, loading capacity: 0.64 mmol/g) is put into a reactor. 400 mL of DCM is added thereto, the resulting mixture is stirred at 20 to 30° C. for 30 minutes, and then the solvent is removed.

### (2) Preparation of Fmoc-D-Ala-NH-Resin

To remove Fmoc, 400 mL of 20% piperidine in DMF is added to the reactor and the resulting mixture is stirred at 20±5° C. for 10 minutes. The procedure is repeated one more time. The reaction solution is removed and a resin is washed with 400 mL of DMF at 20 to 30° C. for 2 minutes. The washing is repeated five more times. Fmoc-D-Ala-OH (12.4 g, 2.0 eq) and HOBt (5.9 g, 2.2 eq) are dissolved in 400 mL of DMF. This solution is put into the reactor, DIC (6.3 mL, 2.0 eq) is added thereto, and then the resulting mixture is stirred at 20±5° C. for 4 hours. After 4 hours of reaction, the reaction solution is removed and the residue is washed with 400 mL of DMF for 2 minutes. The washing is repeated one more time.

### (3) Preparation of Fmoc-Pro-D-Ala-NH-Resin

To remove Fmoc, 400 mL of 20% piperidine in DMF is added to the reactor and the resulting mixture is stirred at 20±5° C. for 10 minutes. The procedure is repeated one more time. The reaction solution is removed and a resin is washed with 400 mL of DMF at 20 to 30° C. for 2 minutes. The washing is repeated five more times. Fmoc-Pro-OH (13.5 g, 2.0 eq) and HOBt (5.9 g, 2.2 eq) are dissolved in 400 mL of DMF. This solution is put into the reactor, DIC (5.0 g, 2.0 eq) is added thereto, and then the resulting mixture is stirred at 20±5° C. for 4 hours. After 4 hours of reaction, the reaction solution is removed and the residue is washed with 400 mL of DMF for 2 minutes. The washing is repeated one more time.

### (4) Preparation of Fmoc-hArg(Et)₂-Pro-D-Ala-NH-Resin

To remove Fmoc, 400 mL of 20% piperidine in DMF is added to the reactor and the resulting mixture is stirred at 20±5° C. for 10 minutes. The procedure is repeated one more time. The reaction solution is removed and a resin is washed with 400 mL of DMF at 20 to 30° C. for 2 minutes. The washing is repeated five more times. Fmoc-hArg(Et)₂—OH (20.1 g, 2.0 eq) and HOBt (5.9 g, 2.2 eq) are dissolved in 400 mL of DMF. This solution is put into the reactor, DIC (5.0 g, 2.0 eq) is added thereto, and then the resulting mixture is stirred at 20±5° C. for 4 hours. After 4 hours of reaction, the reaction solution is removed and the residue is washed with 400 mL of DMF for 2 minutes. The washing is repeated one more time.

### (5) Preparation of Fmoc-Leu-hArg(Et)₂-Pro-D-Ala-NH-Resin

To remove Fmoc, 400 mL of 20% piperidine in DMF is added to the reactor and the resulting mixture is stirred at 20±5° C. for 10 minutes. The procedure is repeated one more time. The reaction solution is removed and a resin is washed with 400 mL of DMF at 20 to 30° C. for 2 minutes. The washing is repeated five more times. Fmoc-Leu-OH (14.1 g, 2.0 eq) and HOBt (5.9 g, 2.2 eq) are dissolved in 400 mL of DMF. This solution is put into the reactor, DIC (5.0 g, 2.0 eq) is added thereto, and then the resulting mixture is stirred at 20±5° C. for 4 hours. After 4 hours of reaction, the reaction solution is removed and the residue is washed with 400 mL of DMF for 2 minutes. The washing is repeated one more time.

### (6) Preparation of Fmoc-D-hArg(Et)₂-Leu-hArg(Et)₂-Pro-D-Ala-NH-Resin

To remove Fmoc, 400 mL of 20% piperidine in DMF is added to the reactor and the resulting mixture is stirred at 20±5° C. for 10 minutes. The procedure is repeated one more time. The reaction solution is removed and a resin is washed with 400 mL of DMF at 20 to 30° C. for 2 minutes. The washing is repeated five more times. Fmoc-D-hArg(Et)₂—OH (20.1 g, 2.0 eq) and HOBt (5.9 g, 2.2 eq) are dissolved in 400 mL of DMF. This solution is put into the reactor, DIC (5.0 g, 2.0 eq) is added thereto, and then the resulting mixture is stirred at 20±5° C. for 4 hours. After 4 hours of reaction, the reaction solution is removed and the residue is washed with 400 L of DMF for 2 minutes. The washing is repeated one more time.

### (7) Preparation of Fmoc-Tyr (tBu)-D-hArg(Et)₂-Leu-hArg(Et)₂-Pro-D-Ala-NH-Resin

To remove Fmoc, 400 mL of 20% piperidine in DMF is added to the reactor and the resulting mixture is stirred at 20±5° C. for 10 minutes. The procedure is repeated one more time. The reaction solution is removed and a resin is washed with 400 mL of DMF at 20 to 30° C. for 2 minutes. The washing is repeated five more times. Fmoc-Tyr (tBu)-OH (18.4 g, 2.0 eq) and HOBt (5.9 g, 2.2 eq) are dissolved in 400 mL of DMF. This solution is put into the reactor, DIC (5.0 g, 2.0 eq) is added thereto, and then the resulting mixture is stirred at 20±5° C. for 4 hours. After 4 hours of reaction, the reaction solution is removed and the residue is washed with 400 mL of DMF for 2 minutes. The washing is repeated one more time. To remove Fmoc, 400 mL of 20% piperidine in DMF is added to the reactor and the resulting mixture is stirred at 20±5° C. for 10 minutes. The procedure is repeated one more time. The reaction solution is removed and a resin is washed with 400 mL of DMF at 20 to 30° C. for 2 minutes. The washing is repeated two more times. The resin is washed with 400 mL of DCM at 20±5° C. for 2 minutes. The washing is repeated two more times. After the resin is dried under nitrogen, a peptide represented by Chemical Formula 5 is obtained (or Chemical Formula 16) (Chemical Formula 16:H₂N-Tyr (R¹)-D-hArg(Et)₂-Leu-hArg(Et)₂-Pro-D-Ala-NH-Resin).

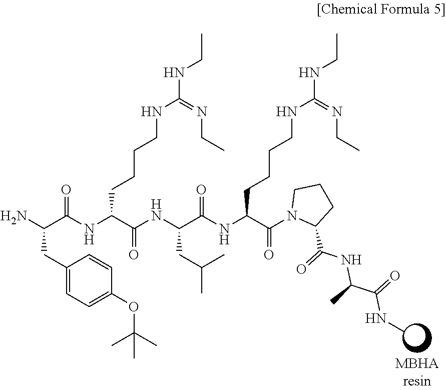 [SMILES: CC/N=C(/NCC)NCCCC[C@H](NC(=O)[C@H](CC(C)C)NC(=O)[C@@H](CCCCN/C(=N\CC)NCC)NC(=O)[C@@H](N)Cc1ccc(OC(C)(C)C)cc1)C(=O)N1CCC[C@@H]1C(=O)N[C@H](C)C(=O)NC]

### (8) Global Cleavage Reaction

600 mL of a cooled cleavage solution (TFA:DCM:H₂O=70%: 29%: 1%, v/v) is slowly added to the dried resin, and the resulting mixture is stirred at 15 to 30° C. for 3 hours. After 3 hours of global cleavage reaction, the completion of the reaction is confirmed by HPLC, and then the cleavage solution is drained and collected. The collected cleavage solution is solidified by slowly adding dropwise 2.5 L of cooled diethyl ether, and stirred for 30 minutes. A solid is isolated from the suspension using a centrifuge and dried to obtain 18 g of GNA C6 of Chemical Formula 6 (TFA salt form, molecular weight: 914.2 g/mol, purity: 82.9%, and yield: 98.4%) (Chemical Formula 17: H₂N-Tyr-D-hArg(Et)₂-Leu-hArg(Et)₂-Pro-D-Ala-NH₂).

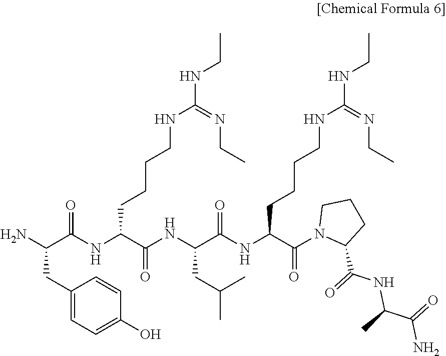 [SMILES: CC/N=C(/NCC)NCCCC[C@H](NC(=O)[C@H](CC(C)C)NC(=O)[C@@H](CCCCN/C(=N\CC)NCC)NC(=O)[C@@H](N)Cc1ccc(O)cc1)C(=O)N1CCC[C@@H]1C(=O)N[C@H](C)C(N)=O]

### 3. GNA Convergent Synthesis

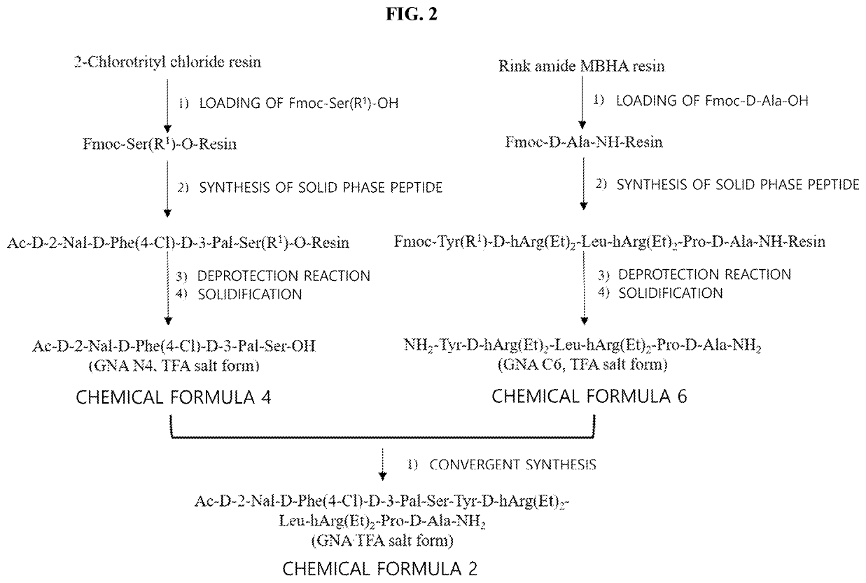

In the reactor, GNA N4 (synthesis scale: 2.55 mmol, 1.72 g, 1.0 eq) is stirred in DMF (15.5 mL) and completely dissolved. GNA C6 (3.0 g, 1.29 eq) and HOAt (0.55 g, 1.58 eq) are added thereto, and the resulting mixture is completely dissolved. After the internal temperature of the reaction solution is cooled to 0° C. using an ice bath, DIEA (0.66 g, 2.0 eq) is added thereto. EDC·HCl (0.77 g, 1.58 eq) is put into DMF (4.5 mL), DIEA (0.66 g, 2.0 eq) is added thereto, and the resulting mixture is completely dissolved. After a solution in which the EDC·HCl is dissolved is slowly added dropwise thereto, the ice bath is removed, and the resulting mixture is stirred for 3 hours. The reaction solution is drained, collected, and slowly added to the mixed solvent (200.0 mL, diethyl ether:DCM=1:1, v/v), and the resulting mixture is stirred at 0 to 3° C. for 30 minutes (reaction solution:mixed solvent=1:10). The mixture is filtered and washed (diethyl ether, 1000 mL×2) to obtain a precipitated solid, and then dried under vacuum (4 hours) to obtain 4.5 g of GNA (TFA salt form, molecular weight: 1570.35 g/mol, purity: 77.3%, yield: 112.5%) of Chemical Formula 2 (Chemical Formula 13: Ac-D-2-Nal-D-Phe(4-Cl)-D-3-Pal-Ser-Tyr-D-hArg(Et)₂-Leu-hArg(Et)₂-Pro-D-Ala-NH₂). A flowchart of the entire process is shown in FIG. 2.

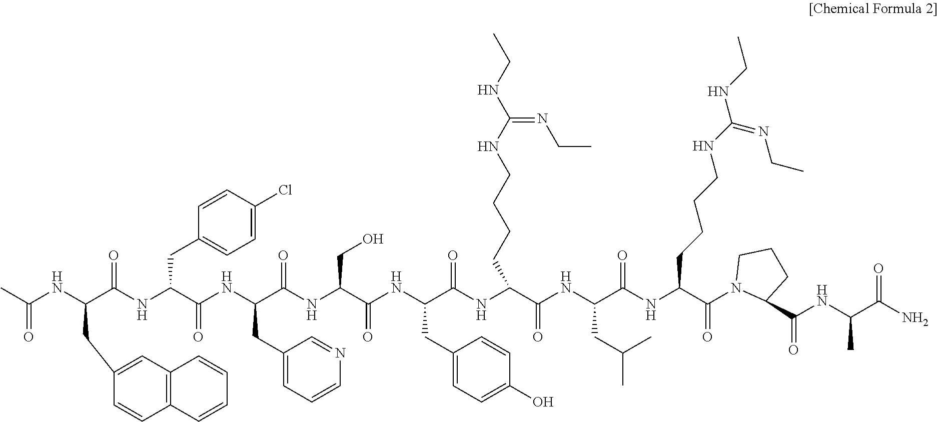 [SMILES: CC/N=C(/NCC)NCCCC[C@H](NC(=O)[C@H](CC(C)C)NC(=O)[C@@H](CCCCN/C(=N\CC)NCC)NC(=O)[C@H](Cc1ccc(O)cc1)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N1CCC[C@H]1C(=O)N[C@H](C)C(N)=O]

### Example 3. Manufacture of Ganirelix by [5+5] Convergent Synthesis

### 1. GNAN5

### (1) Swelling

67.6 g of a 2-chlorotrityl chloride resin (100 mmol, loading capacity: 1.48 mmol/g) is put into a reactor. 1.25 L of DCM is added thereto, the resulting mixture is stirred at 20 to 30° C. for 30 minutes, and then the solvent is removed.

### (2) Preparation of Fmoc-Tyr (tBu)-O-Resin

Fmoc-Tyr (tBu)-OH (114.9 g, 2.5 eq) and DIEA (51.7 g, 4.0 eq) are dissolved in 1.25 L of DCM. After this solution is added to the reactor, the resulting mixture is stirred at 20 to 30° C. for 4 hours. The reaction solution is removed and the residue is washed with 1.25 L of DMF at 20 to 30° C. for 2 minutes. The washing is repeated one more time. 1.25 L of a capping solution (DCM:MeOH:DIEA=85:10:5, v/v) is added to the reactor and the resulting mixture is stirred at 20 to 30° C. for 15 minutes. The capping is repeated one more time. The reaction solution is removed and a resin is washed with 1.25 L of DMF at 20 to 30° C. for 2 minutes. The washing is repeated one more time. The resin is washed with 1.25 L of DMF at 20 to 30° C. for 2 minutes. The washing is repeated one more time.

### (3) Preparation of Fmoc-Ser(tBu)-Tyr (tBu)-O-Resin

To remove Fmoc, 1.25 L of 20% piperidine in DMF is added to the reactor and the resulting mixture is stirred at 20±5° C. for 15 minutes. The procedure is repeated one more time. The reaction solution is removed and a resin is washed with 1.25 L of DMF at 20 to 30° C. for 2 minutes. The washing is repeated two more times. The resin is washed with 1.25 L of DCM at 20 to 30° C. for 2 minutes. The washing is repeated one more time. The resin is washed with 1.25 L of DMF at 20 to 30° C. for 2 minutes. Fmoc-Ser(tBu)-OH (64.8 g, 2.5 eq), HOBt (25.1 g, 2.75 eq), and DIEA (8.7 g, 1.0 eq) are dissolved in 845 mL of DMF. This solution is put into the reactor, DIC (21.3 g, 2.5 eq) is added thereto, and then the resulting mixture is stirred at 20±5° C. for 2 hours. The reaction solution is removed and the residue is washed with 845 mL of DMF for 2 minutes. The washing is repeated one more time.

### (4) Preparation of Fmoc-D-3-Pal-Ser(tBu)-Tyr (tBu)-O-Resin

To remove Fmoc, 845 mL of 20% piperidine in DMF is added to the reactor and the resulting mixture is stirred at 20±5° C. for 15 minutes. The procedure is repeated one more time. The reaction solution is removed and a resin is washed with 845 mL of DMF at 20 to 30° C. for 2 minutes. The washing is repeated two more times. The resin is washed with 845 mL of DCM at 20 to 30° C. for 2 minutes. The washing is repeated one more time. The resin is washed with 845 mL of DMF 845 mL at 20 to 30° C. for 2 minutes. Fmoc-D-3-Pal-OH (52.5 g, 2.0 eq) and HOBt (20.1 g, 2.2 eq) are dissolved in 675 mL of DMF. This solution is put into the reactor, DIC (17.1 g, 2.0 eq) is added thereto, and then the resulting mixture is stirred at 20±5° C. for 2 hours. The reaction solution is removed and the residue is washed with 675 mL of DMF for 2 minutes. The washing is repeated one more time.

### (5) Preparation of Fmoc-D-Phe(4-Cl)-D-3-Pal-Ser(tBu)-Tyr (tBu)-O-Resin

To remove Fmoc, 675 mL of 20% piperidine in DMF is added to the reactor and the resulting mixture is stirred at 20±5° C. for 15 minutes. The procedure is repeated one more time. The reaction solution is removed and a resin is washed with 675 mL of DMF at 20 to 30° C. for 2 minutes. The washing is repeated two more times. The resin is washed with 675 mL of DCM at 20 to 30° C. for 2 minutes. The washing is repeated one more time. The resin is washed with 675 mL of DMF 845 mL at 20 to 30° C. for 2 minutes. Fmoc-D-Phe(4-Cl)—OH (57.0 g, 2.0 eq) and HOBt (20.1 g, 2.2 eq) are dissolved in 675 mL of DMF. This solution is put into the reactor, DIC (17.1 g, 2.0 eq) is added thereto, and then the resulting mixture is stirred at 20±5° C. for 2 hours. The reaction solution is removed and the residue is washed with 675 mL of DMF for 2 minutes. The washing is repeated one more time.

### (6) Preparation of Fmoc-D-2-Nal-D-Phe(4-Cl)-D-3-Pal-Ser(tBu)-Tyr (tBu)-O-Resin

To remove Fmoc, 675 mL of 20% piperidine in DMF is added to the reactor and the resulting mixture is stirred at 20±5° C. for 15 minutes. The procedure is repeated one more time. The reaction solution is removed and a resin is washed with 675 mL of DMF at 20 to 30° C. for 2 minutes. The washing is repeated two more times. The resin is washed with 675 mL of DCM at 20 to 30° C. for 2 minutes. The washing is repeated one more time. The resin is washed with 675 mL of DMF 845 mL at 20 to 30° C. for 2 minutes. Fmoc-D-2-Nal-OH (59.1 g, 2.0 eq) and HOBt (20.1 g, 2.2 eq) are dissolved in 675 mL of DMF. This solution is put into the reactor, DIC (17.1 g, 2.0 eq) is added thereto, and then the resulting mixture is stirred at 20±5° C. for 2 hours. The reaction solution is removed and the residue is washed with 675 mL of DMF for 2 minutes. The washing is repeated one more time.

### (7) Preparation of Ac-D-2-Nal-D-Phe(4-Cl)-D-3-Pal-Ser(tBu)-Tyr (tBu)-O-Resin

To remove Fmoc, 675 mL of 20% piperidine in DMF is added to the reactor and the resulting mixture is stirred at 20±5° C. for 15 minutes. The procedure is repeated one more time. The reaction solution is removed and a resin is washed with 675 mL of DMF at 20 to 30° C. for 2 minutes. The washing is repeated two more times. The resin is washed with 675 mL of DCM at 20 to 30° C. for 2 minutes. The washing is repeated one more time. The resin is washed with 675 mL of DMF 845 mL at 20 to 30° C. for 2 minutes. DIEA (25.9 g, 2.0 eq) is dissolved in 675 mL of DMF and put into the reactor, acetic anhydride (20.4 g, 2.0 eq) is added thereto, and then the resulting mixture is stirred at 20±5° C. for 2 hours. The reaction solution is removed and the residue is washed with DMF (675 mL) at 20±5° C. for 2 minutes. The washing is repeated two more times. The residue is washed with DCM (675 mL) at 20±5° C. for 2 minutes. The washing is repeated two more times. After the resin is dried under nitrogen, a peptide represented by Chemical Formula 7 (or Chemical Formula 18) is obtained (Chemical Formula 18: Ac-D-2-Nal-D-Phe(4-Cl)-D-3-Pal-Ser (R¹)-Tyr (R¹)—O-Resin).

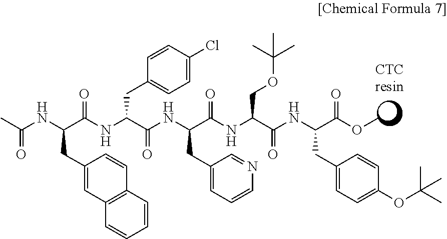 [SMILES: COC(=O)[C@H](Cc1ccc(OC(C)(C)C)cc1)NC(=O)[C@H](COC(C)(C)C)NC(=O)[C@@H](Cc1cccnc1)NC(=O)[C@@H](Cc1ccc(Cl)cc1)NC(=O)[C@@H](Cc1ccc2ccccc2c1)NC(C)=O]

### (8) Global Cleavage Reaction for Preparation of GNA N5 (TFA Salt)

3.0 mL of a cooled cleavage solution (TFA:TIS:H₂O=95%: 2.5%: 2.5%, v/v) is slowly added to the dried resin, and the resulting mixture is stirred at 15 to 30° C. for 3 hours. After 2 hours of global cleavage reaction, the completion of the reaction is confirmed through HPLC analysis, and then the cleavage solution is drained and collected. The collected cleavage solution is concentrated to 1 L under reduced pressure. 10 L of diethyl ether (cleavage reaction concentrated solution:diethyl ether=1:10) cooled to 10° C. is solidified by slowly adding dropwise the concentrated cleavage solution, and stirred for 10 hour. The suspension was filtered under reduced pressure, washed with 5 L of diethyl ether, and dried under nitrogen for 24 hours or more to obtain 104.4 g of GNA N5 of Chemical Formula 8 (TFA salt form, molecular weight: 837.33 g/mol, purity: 94.7%, and yield: 124.7%) (Chemical Formula 19: Ac-D-2-Nal-D-Phe (4-Cl)-D-3-Pal-Ser-Tyr-OH).

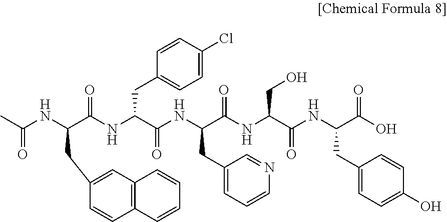 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(O)cc1)C(=O)O]

### 2. GNA C5

### (1) Swelling

15.9 g of a rink amide MBHA resin (10 mmol, loading capacity: 0.631 mmol/g) is put into a reactor. 200 L of DCM is added thereto, the resulting mixture is stirred at 20 to 30° C. for 30 minutes, and then the solvent is removed.

### (2) Preparation of Fmoc-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20 to 30° C. for 2 minutes. The washing is repeated five more times. Fmoc-D-Ala-OH (7.9 g, 2.5 eq) and HOBt (3.8 g, 2.75 eq) are dissolved in 250 mL of DMF 100. This solution is put into the reactor, DIC (3.2 g, 2.5 eq) is added thereto, and then the resulting mixture is stirred at 20±5° C. for 4 hours. After 4 hours of reaction, the reaction solution is removed and the residue is washed with 100 mL of DMF for 2 minutes. The washing is repeated one more time.

### (3) Preparation of Fmoc-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20 to 30° C. for 2 minutes. The washing is repeated five more times. Fmoc-Pro-OH (8.5 g, 2.5 eq) and HOBt (3.8 g, 2.75 eq) are dissolved in 250 ml of DMF. This solution is put into the reactor, DIC (3.2 g, 2.5 eq) is added thereto, and then the resulting mixture is stirred at 20±5° C. for 3 hours. After 3 hours of reaction, the reaction solution is removed and the residue is washed with 100 mL of DMF for 2 minutes. The washing is repeated one more time.

### (4) Preparation of Fmoc-hArg(Et)₂-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20 to 30° C. for 2 minutes. The washing is repeated five more times. Fmoc-hArg(Et)₂—OH (12.7 g, 2.5 eq) and HOBt (3.8 g, 2.75 eq) are dissolved in 250 mL of DMF. This solution is put into the reactor, DIC (3.2 g, 2.5 eq) is added thereto, and then the resulting mixture is stirred at 20±5° C. for 3 hours. After 3 hours of reaction, the reaction solution is removed and the residue is washed with 100 mL of DMF for 2 minutes. The washing is repeated one more time.

### (5) Preparation of Fmoc-Leu-hArg(Et)₂-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20 to 30° C. for 2 minutes. The washing is repeated five more times. Fmoc-Leu-OH (8.9 g, 2.5 eq) and HOBt (3.8 g, 2.75 eq) are dissolved in 250 mL of DMF. This solution is put into the reactor, DIC (3.2 g, 2.5 eq) is added thereto, and then the resulting mixture is stirred at 20±5° C. for 3 hours. After 3 hours of reaction, the reaction solution is removed and the residue is washed with 100 mL of DMF for 2 minutes. The washing is repeated one more time.

### (6) Preparation of Fmoc-D-hArg(Et)₂-Leu-hArg(Et)₂-Pro-D-Ala-NH-Resin

To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20 to 30° C. for 2 minutes. The washing is repeated five more times. Fmoc-D-hArg(Et)₂—OH (12.7 g, 2.5 eq) and HOBt (3.8 g, 2.75 eq) are dissolved in 250 mL of DMF. This solution is put into the reactor, DIC (3.2 g, 2.5 eq) is added thereto, and then the resulting mixture is stirred at 20±5° C. for 3 hours. After 3 hours of reaction, the reaction solution is removed and the residue is washed with 100 mL of DMF for 2 minutes. The washing is repeated one more time. To remove Fmoc, 100 mL of 20% piperidine in DMF is added to the reactor and the resulting mixture is stirred at 20±5° C. for 15 minutes. The procedure is repeated one more time. The reaction solution is removed and a resin is washed with 100 mL of DMF at 20±5° C. for 2 minutes. The washing is repeated five more times. The resin is washed with 100 mL of DCM at 20±5° C. for 2 minutes. The washing is repeated two more times. After the resin is dried under nitrogen, a peptide represented by Chemical Formula 9 (or Chemical Formula 20) is obtained (Chemical Formula 20: H₂N-D-hArg(Et)₂-Leu-hArg(Et)₂-Pro-D-Ala-NH-Resin).

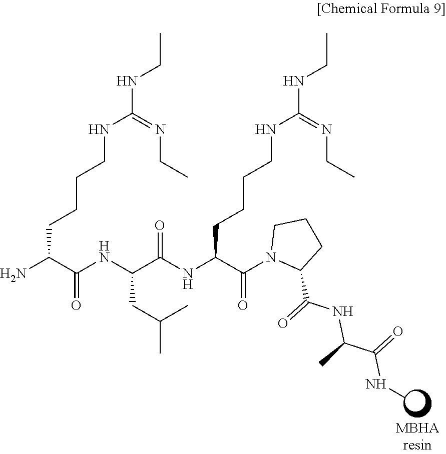 [SMILES: CC/N=C(/NCC)NCCCC[C@@H](N)C(=O)N[C@@H](CC(C)C)C(=O)N[C@@H](CCCCN/C(=N\CC)NCC)C(=O)N1CCC[C@@H]1C(=O)N[C@H](C)C(=O)NC]

### (7) Global Cleavage Reaction for Preparation of GNA C5 (TFA Salt)

300 mL of a cooled cleavage solution (TFA:DCM:H₂O=70:27.5:2.5, v/v) is slowly added to the dried resin, and the resulting mixture is stirred at 15 to 30° C. for 3 hours. After 3 hours of global cleavage reaction, the completion of the reaction is confirmed by HPLC, and then the cleavage solution is drained and collected. The collected cleavage solution is concentrated to 30 mL under reduced pressure. 600 mL of diethyl ether (cleavage reaction concentrated solution:diethyl ether=1:20) cooled to −5° C. is solidified by slowly adding dropwise the concentrated cleavage solution, and stirred for 30 minutes. The suspension was filtered under reduced pressure, washed twice with 200 mL of diethyl ether, and then dried to obtain 11.2 g of GNA C5 of Chemical Formula 10 (TFA salt form, molecular weight: 751.04 g/mol, purity: 82.9%, and yield: 149.3%) (Chemical Formula 21: H₂N-D-hArg(Et)₂-Leu-hArg(Et)₂-Pro-D-Ala-NH₂).

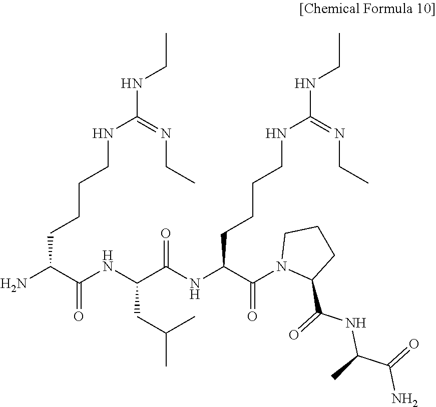 [SMILES: CC/N=C(/NCC)NCCCC[C@@H](N)C(=O)N[C@@H](CC(C)C)C(=O)N[C@@H](CCCCN/C(=N\CC)NCC)C(=O)N1CCC[C@H]1C(=O)N[C@H](C)C(N)=O]

### 3. GNA Convergent Synthesis

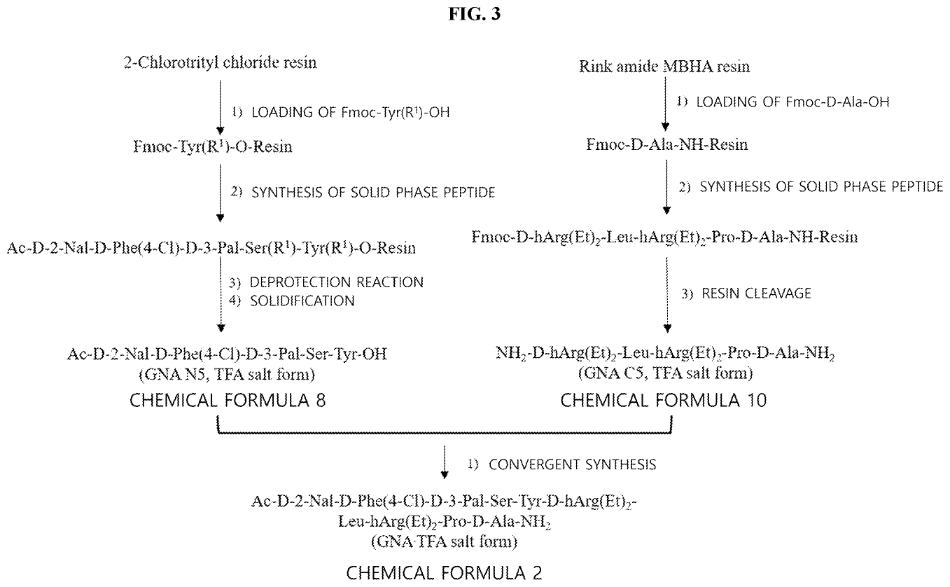

GNA N5 (synthesis scale: 3.7 mmol, 3.1 g, 1.0 eq) and HOAt (0.67 g, 1.33 eq) are dissolved in 26 mL of DMF. GNA C5 (4.28 g, 1.54 eq) is added thereto to dissolve the resulting mixture, DIEA (0.42 g, 0.88 eq) is slowly added thereto, and the internal temperature of the reaction solution is cooled to 0 to 3° C. using an ice bath. EDC·HCl (1.25 g, 1.76 eq) and DIEA (0.84 g, 1.76 eq) are completely dissolved in 6.5 mL of DCM and the temperature inside the solution is cooled to 0 to 3° C. using an ice bath. After a DCM solution of dissolved EDC·HCl is slowly added to the reaction solution at 0 to 3° C., the resulting mixture is stirred at 10 to 25° C. for 1 hour or more. The reaction solution is drained and collected, the reaction solution is added to 325 mL of an MTBE:DCM=1:1 (v/v) solution cooled to 10° C. or less, and the resulting mixture is stirred for 30 minutes. To obtain a precipitated solid, the solution is filtered under reduced pressure, and the filtered product is washed with 160 mL of an MTBE:DCM=1:1 (v/v) solution, and then dried under nitrogen for 15 hours or more to obtain 6.3 g of GNA (TFA salt form, molecular weight: 1570.35 g/mol, purity: 84.4%, and yield: 108.6%) of Chemical Formula 2 (Chemical Formula 13: Ac-D-2-Nal-D-Phe(4-Cl)-D-3-Pal-Ser-Tyr-D-hArg(Et)₂-Leu-hArg(Et)₂-Pro-D-Ala-NH₂). A flowchart of the entire process is shown in FIG. 3.

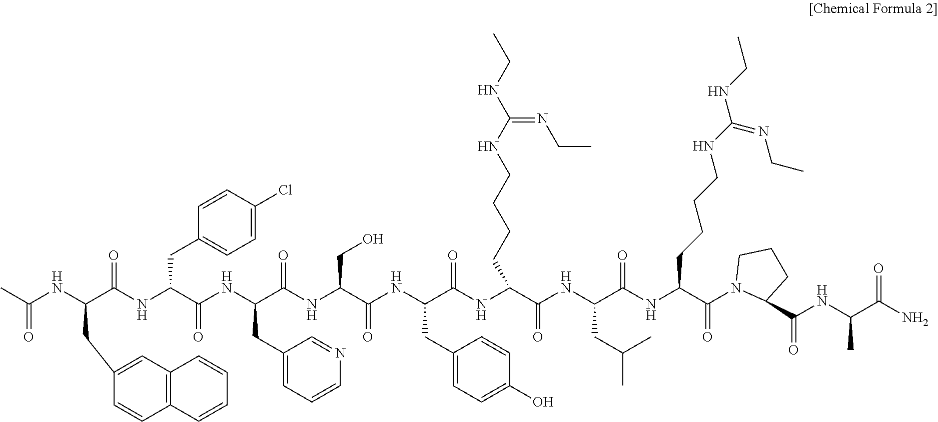 [SMILES: CC/N=C(/NCC)NCCCC[C@H](NC(=O)[C@H](CC(C)C)NC(=O)[C@@H](CCCCN/C(=N\CC)NCC)NC(=O)[C@H](Cc1ccc(O)cc1)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N1CCC[C@H]1C(=O)N[C@H](C)C(N)=O]

The experimental results of Examples 1 to 3 are summarized in the following Table 1. Among them, the experimental results of Example 3 were determined to be the most excellent, and subsequent research was conducted on this.

TABLE 1

| | | | Crude product | |
|---|---|---|---|---|
| | Synthesis method | Scale | Synthesis yield | Purity |
| 1 | Linear synthesis | 10 mmol | 90.1% | 46.7% |
| 2 | [4 + 6] convergent synthesis | 2.55 mmol | 112.5% | 77.3% |
| 3 | [5 + 5] convergent synthesis | 3.7 mmol | 108.6% | 84.4% |

### Experimental Example 1. Research on Intermediate Morphology

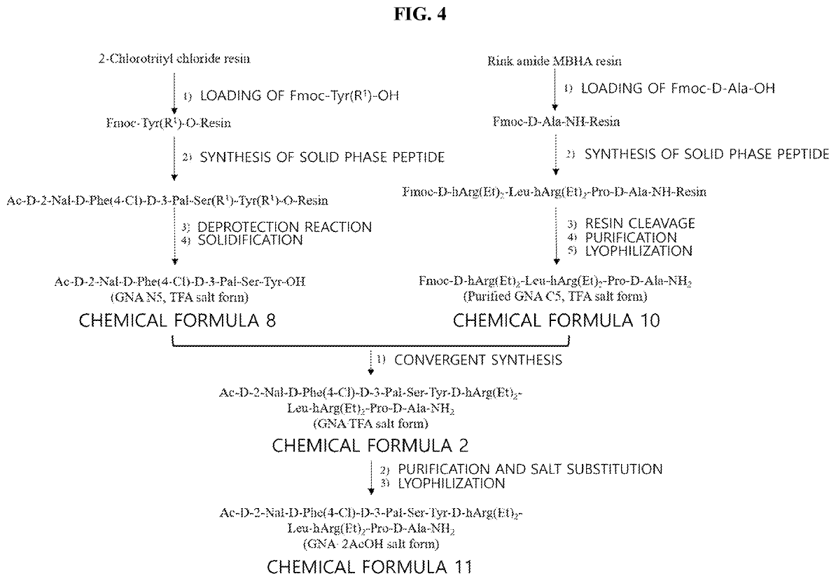

The morphology of each intermediate was determined by evaluating the purity of ganirelix (GNA, TFA form) during convergent synthesis depending on whether or not the intermediates (GNA N5, GNA C5) were purified. In the case of GNA N5, when a crude product or a purified product was used, ganirelix (GNA, TFA form) of similar purity was obtained (the following Table 2, Experiments 1 and 2/Experiments 3 and 4). In contrast, in the case of GNA C5, higher purity ganirelix (GNA, TFA form) was obtained than when a purified product was used (the following Table 2, Experiments 1 and 3/Experiments 2 and 4). Therefore, in the following subsequent research, a manufacturing process was established using convergent synthesis for GNA N5 as a crude product and GNAC5 as a purified product, and the process flow diagram in this regard is shown in FIG. 4.

TABLE 2

| | | Ganirelix | |
|---|---|---|---|
| Exper- | | Synthesis | |
| iment | Intermediate | yield | Purity |
| 1 | GNA C5 (purified product; purity 99.5%) | 125.0% | 96.6% |
| | GNA N5 (purified product; purity 98.8%) | | |
| 2 | GNA C5 (purified product; purity 99.5%) | 118.8% | 97.2% |
| | GNA N5 (crude product; purity 96.4%) | | |
| 3 | GNA C5 (crude product; purity 92.3%) | 131.3% | 93.8% |
| | GNA N5 (purified product; 98.8%) | | |
| 4 | GNA C5 (crude product; 90.4%) | 127.7% | 92.1% |
| | GNA N5 (crude product; purity 96.7%) | | |

### Experimental Example 2. Confirmation of Possibility of Mass Synthesis of GNA N5

### 1. Swelling

68.5 g of a 2-chlorotrityl chloride resin (100 mmol, loading capacity: 1.46 mmol/g) is put into a reactor. 2.0 L (20 L/mol) of DCM is added thereto, the resulting mixture is stirred at 20 to 30° C. for 30 minutes, and then the solvent is removed.

### 2. Preparation of Fmoc-Tyr (tBu)-O-Resin

Fmoc-Tyr (tBu)-OH (160.8 g, 3.5 eq) and DIEA (90.5 g, 7.0 eq) are dissolved in 2.0 L of DCM. After this solution is added to the reactor, the resulting mixture is stirred at 20 to 30° C. for 4 hours. The reaction solution is removed and the residue is washed with 2.0 L of DMF at 20 to 30° C. for 2 minutes. The washing is repeated one more time. 2.0 L of a capping solution (DCM:MeOH:DIEA=85:10:5, v/v) is added to the reactor and the resulting mixture is stirred at 20 to 30° C. for 15 minutes. The capping is repeated one more time. The reaction solution is removed and a resin is washed with 2.0 L of DMF at 20 to 30° C. for 2 minutes. The washing is repeated one more time. The resin is washed with 2.0 L of DMF at 20 to 30° C. for 2 minutes. The washing is repeated one more time.

### 3. Preparation of Fmoc-Ser(tBu)-Tyr (tBu)-O-Resin

To remove Fmoc, 2.0 L of 20% piperidine in DMF is added to the reactor and the resulting mixture is stirred at 20±5° C. for 15 minutes. The procedure is repeated one more time. The reaction solution is removed and a resin is washed with 2.0 L of DMF at 20 to 30° C. for 2 minutes. The washing is repeated five more times. Fmoc-Ser(tBu)-OH (95.9 g, 2.5 eq), Oxyma (39.1 g, 2.75 eq), and DIEA (19.4 g, 1.5 eq) are dissolved in 2.0 L of DMF. This solution is put into the reactor, DIC (31.6 g, 2.5 eq) is added thereto, and then the resulting mixture is stirred at 20±5° C. for 3 hours. The reaction solution is removed and the residue is washed with 2.0 L of DMF for 2 minutes. The washing is repeated one more time.

### 4. Preparation of Fmoc-D-3-Pal-Ser(tBu)-Tyr (tBu)-O-Resin

To remove Fmoc, 2.0 L of 20% piperidine in DMF is added to the reactor and the resulting mixture is stirred at 20±5° C. for 15 minutes. The procedure is repeated one more time. The reaction solution is removed and a resin is washed with 2.0 L of DMF at 20±5° C. for 2 minutes. The washing is repeated five more times. Fmoc-D-3-Pal-OH (97.1 g, 2.5 eq) and Oxyma (39.1 g, 2.75 eq) are dissolved in 2.0 L of DMF. This solution is put into the reactor, DIC (31.6 g, 2.5 eq) is added thereto, and then the resulting mixture is stirred at 20±5° C. for 3 hours. The reaction solution is removed and the residue is washed with 2.0 L of DMF for 2 minutes. The washing is repeated one more time.

### 5. Preparation of Fmoc-D-Phe(4-Cl)-D-3-Pal-Ser(tBu)-Tyr (tBu)-O-Resin

To remove Fmoc, 2.0 L of 20% piperidine in DMF is added to the reactor and the resulting mixture is stirred at 20±5° C. for 15 minutes. The procedure is repeated one more time. The reaction solution is removed and a resin is washed with 2.0 L of DMF at 20±5° C. for 2 minutes. The washing is repeated five more times. Fmoc-D-Phe (4-Cl)—OH (105.5 g, 2.5 eq) and Oxyma (39.1 g, 2.75 eq) are dissolved in 2.0 L of DMF. This solution is put into the reactor, DIC (31.6 g, 2.5 eq) is added thereto, and then the resulting mixture is stirred at 20±5° C. for 3 hours. The reaction solution is removed and the residue is washed with 2.0 L of DMF for 2 minutes. The washing is repeated one more time.

### 6. Preparation of Fmoc-D-2-Nal-D-Phe(4-Cl)-D-3-Pal-Ser(tBu)-Tyr (tBu)-O-Resin

To remove Fmoc, 2.0 L of 20% piperidine in DMF is added to the reactor and the resulting mixture is stirred at 20±5° C. for 15 minutes. The procedure is repeated one more time. The reaction solution is removed and a resin is washed with 2.0 L of DMF at 20±5° C. for 2 minutes. The washing is repeated five more times. Fmoc-D-2-Nal-OH (109.4 g, 2.5 eq) and Oxyma (39.1 g, 2.75 eq) are dissolved in 2.0 L of DMF. This solution is put into the reactor, DIC (31.6 g, 2.5 eq) is added thereto, and then the resulting mixture is stirred at 20±5° C. for 3 hours. The reaction solution is removed and the residue is washed with 2.0 L of DMF for 2 minutes. The washing is repeated one more time.

### 7. Preparation of Ac-D-2-Nal-D-Phe(4-Cl)-D-3-Pal-Ser(tBu)-Tyr (tBu)-O-Resin

To remove Fmoc, 2.0 L of 20% piperidine in DMF is added to the reactor and the resulting mixture is stirred at 20±5° C. for 15 minutes. The procedure is repeated one more time. The reaction solution is removed and a resin is washed with 2.0 L of DMF at 20±5° C. for 2 minutes. The washing is repeated five more times. DIEA (32.3 g, 2.5 eq) is dissolved in 2.0 L of DMF and put into the reactor, acetic anhydride (25.5 g, 2.5 eq) is added thereto, and then the resulting mixture is stirred at 20±5° C. for 2 hours. The reaction solution is removed and the residue is washed with DMF (2.0 L) at 20±5° C. for 2 minutes. The washing is repeated two more times. The residue is washed with DCM (2.0 L) at 20±5° C. for 2 minutes. The washing is repeated two more times. The resin is dried under vacuum.

### 8. Global Cleavage Reaction for Preparation of GNA N5 (TFA Salt)

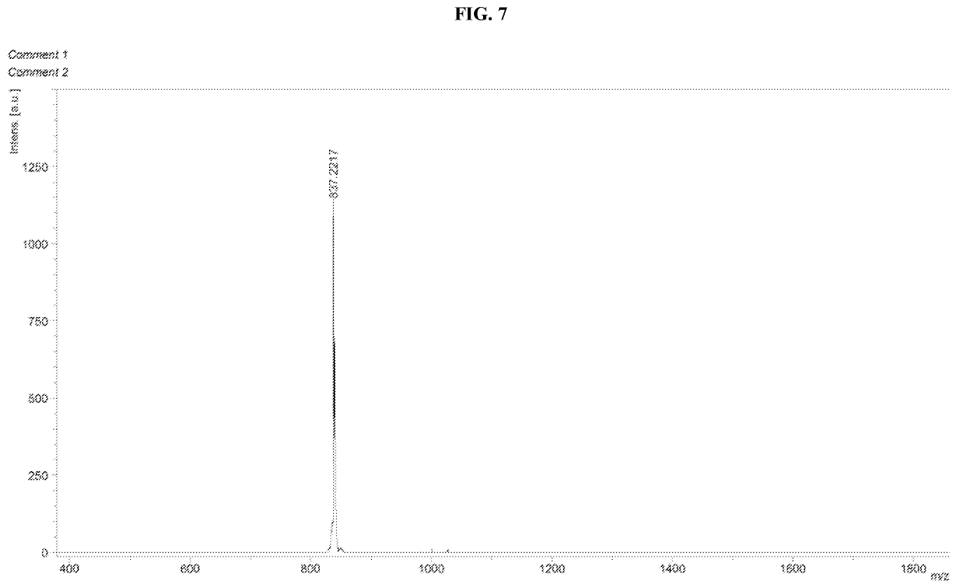

3.0 L of a cooled cleavage solution (TFA:TIS:H₂O=95:2.5:2.5, v/v) is slowly added to the dried resin, and the resulting mixture is stirred at 15 to 30° C. for 3 hours. After 3 hours of global cleavage reaction, the completion of the reaction is confirmed through HPLC analysis, and then the cleavage solution is drained and collected. The collected cleavage solution is concentrated to 30% or less under reduced pressure. The concentrated cleavage solution is solidified by adding dropwise 15 L of cooled MTBE (cleavage reaction solution:MTBE=1:5) and stirred for 30 minutes. The suspension is filtered under reduced pressure, washed with 5.0 L of MTBE (5.0 L×3 times), and then dried under nitrogen for 15 hours or more. It was confirmed that GNA N5 3 batch was reproducibly prepared with an obtained amount (96.8 g, 98.0 g, and 83.6 g), a synthesis yield (115.7%, 117.1%, and 99.9%) and a crude purity (96.3%, 96.4%, and 94.8%) (see the following Table 3). In this case, the molecular weight of the synthesized GNA N5 is 837.33 g/mol (monoisotopic mass: 836.29 g/mol). The molecular weight [M+H]+ measured by a MALDI-TOF mass apparatus is 837.22 g/mol (see FIG. 7).

TABLE 3

| | Synthetically | | |
|---|---|---|---|
| | obtained amount | Synthesis yield | Purity |
| 1^{st} batch | 96.8 g | 115.7% | 96.3% |
| 2^{nd} batch | 98.0 g | 117.1% | 96.4% |
| 3^{rd} batch | 83.6 g | 99.9% | 94.8% |

** Synthesis scale: 100 mmol

### Experimental Example 3. Confirmation of Possibility of Mass Synthesis of GNA C5

### 1. Swelling

153.8 g of a rink amide MBHA resin (100 mmol, loading capacity: 0.65 mmol/g) is put into a reactor. 2.0 L (20 L/mol) of DCM is added thereto, the resulting mixture is stirred at 20 to 30° C. for 30 minutes, and then the solvent is removed.

### 2. Preparation of Fmoc-D-Ala-NH-Resin

To remove Fmoc, 2.0 L of 20% piperidine in DMF is added to the reactor and the resulting mixture is stirred at 20±5° C. for 15 minutes. The procedure is repeated one more time. The reaction solution is removed and a resin is washed with 2.0 L of DMF at 20 to 30° C. for 2 minutes. The washing is repeated five more times. Fmoc-D-Ala-OH (93.4 g, 3.0 eq) and Oxyma (46.9 g, 3.3 eq) are dissolved in 2.0 L of DMF. This solution is put into the reactor, DIC (37.9 g, 3.0 eq) is added thereto, and then the resulting mixture is stirred at 20±5° C. for 3 hours. After 3 hours of reaction, the reaction solution is removed and the residue is washed with 2.0 L of DMF for 2 minutes. The washing is repeated one more time.

### 3. Preparation of Fmoc-Pro-D-Ala-NH-Resin

To remove Fmoc, 2.0 L of 20% piperidine in DMF is added to the reactor and the resulting mixture is stirred at 20±5° C. for 15 minutes. The procedure is repeated one more time. The reaction solution is removed and a resin is washed with 2.0 L of DMF at 20 to 30° C. for 2 minutes. The washing is repeated five more times. Fmoc-Pro-OH (101.2 g, 3.0 eq) and Oxyma (46.9 g, 3.3 eq) are dissolved in 2.0 L of DMF. This solution is put into the reactor, DIC (37.9 g, 3.0 eq) is added thereto, and then the resulting mixture is stirred at 20±5° C. for 3 hours. After 3 hours of reaction, the reaction solution is removed and the residue is washed with 2.0 L of DMF for 2 minutes. The washing is repeated one more time.

### 4. Preparation of Fmoc-hArg(Et)₂-Pro-D-Ala-NH-Resin

To remove Fmoc, 2.0 L of 20% piperidine in DMF is added to the reactor and the resulting mixture is stirred at 20±5° C. for 15 minutes. The procedure is repeated one more time. The reaction solution is removed and a resin is washed with 2.0 L of DMF at 20±5° C. for 2 minutes. The washing is repeated five more times. Fmoc-hArg(Et)₂—OH (150.9 g, 3.0 eq) and Oxyma (46.9 g, 3.3 eq) are dissolved in 2.0 L of DMF. This solution is put into the reactor, DIC (37.9 g, 3.0 eq) is added thereto, and then the resulting mixture is stirred at 20±5° C. for 3 hours. After 3 hours of reaction, the reaction solution is removed and the residue is washed with 2.0 L of DMF for 2 minutes. The washing is repeated one more time.

### 5. Preparation of Fmoc-Leu-hArg(Et)₂-Pro-D-Ala-NH-Resin

To remove Fmoc, 2.0 L of 20% piperidine in DMF is added to the reactor and the resulting mixture is stirred at 20±5° C. for 15 minutes. The procedure is repeated one more time. The reaction solution is removed and a resin is washed with 2.0 L of DMF at 20±5° C. for 2 minutes. The washing is repeated five more times. Fmoc-Leu-OH (106.0 g, 3.0 eq) and Oxyma (46.9 g, 3.3 eq) are dissolved in 2.0 L of DMF. This solution is put into the reactor, DIC (37.9 g, 3.0 eq) is added thereto, and then the resulting mixture is stirred at 20±5° C. for 3 hours. After 3 hours of reaction, the reaction solution is removed and the residue is washed with 2.0 L of DMF for 2 minutes. The washing is repeated one more time.

### 6. Preparation of Fmoc-D-hArg(Et)₂-Leu-hArg(Et)₂-Pro-D-Ala-NH-Resin

To remove Fmoc, 2.0 L of 20% piperidine in DMF is added to the reactor and the resulting mixture is stirred at 20±5° C. for 15 minutes. The procedure is repeated one more time. The reaction solution is removed and a resin is washed with 2.0 L of DMF at 20±5° C. for 2 minutes. The washing is repeated five more times. Fmoc-D-hArg(Et)₂—OH (150.9 g, 3.0 eq) and Oxyma (46.9 g. 3.3 eq) are dissolved in 2.0 L of DMF. This solution is put into the reactor, DIC (37.9 g, 3.0 eq) is added thereto, and then the resulting mixture is stirred at 20±5° C. for 3 hours. After 3 hours of reaction, the reaction solution is removed and the residue is washed with 2.0 L of DMF for 2 minutes. The washing is repeated one more time. To remove Fmoc, 2.0 L of 20% piperidine in DMF is added to the reactor and the resulting mixture is stirred at 20±5° C. for 15 minutes. The procedure is repeated one more time. The reaction solution is removed and the residue is washed with DMF (2.0 L) at 20±5° C. for 2 minutes. The washing is repeated two more times. The residue is washed with DCM (2.0 L) at 20±5° C. for 2 minutes. The washing is repeated two more times. The resin is dried under vacuum.

### 7. Global Cleavage Reaction for Preparation of GNA C5 (TFA Salt)

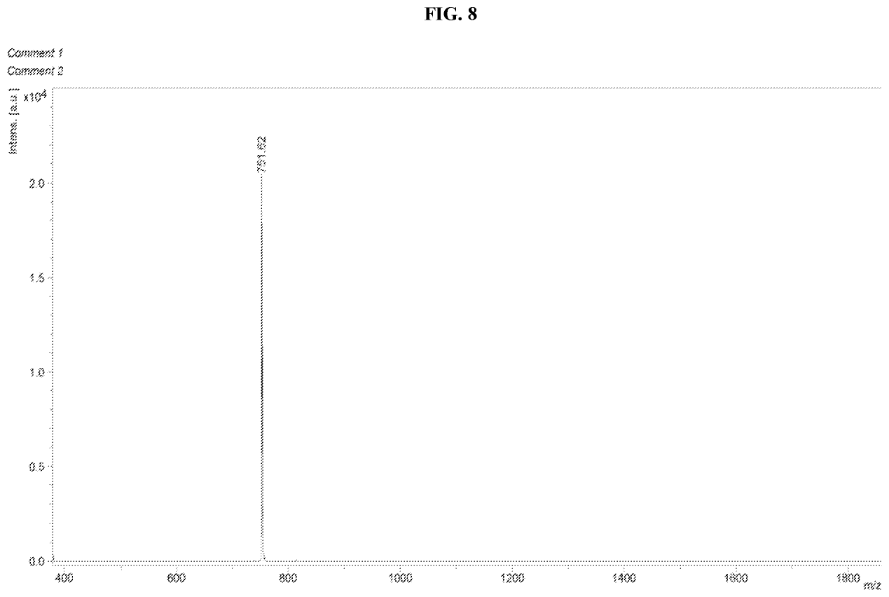

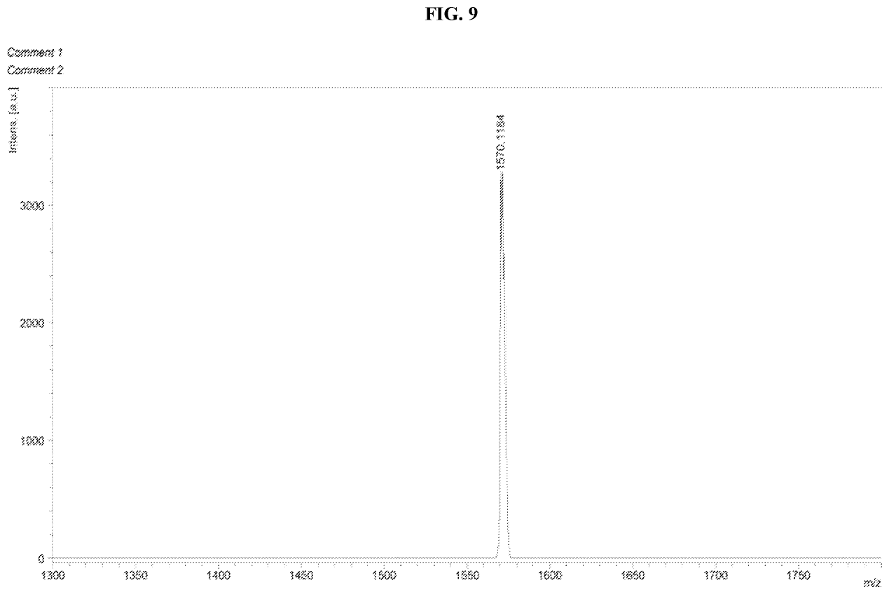

3.0 L of a cooled cleavage solution (TFA:DCM=4:6, v/v) is slowly added to the dried resin, and the resulting mixture is stirred at 15 to 30° C. for 3 hours. After 2 hours of global cleavage reaction, the completion of the reaction is confirmed by HPLC, and then the cleavage solution is drained and collected. 3.0 L of a cleavage reaction solution is slowly added to 30 L of MTBE cooled to 5° C. (cleavage reaction solution:MTBE=1:10), and the resulting mixture is stirred for 30 minutes. To obtain a precipitated solid, the solution is filtered under reduced pressure, and the filtered product is washed three times with 3.5 L of MTBE, and then dried under nitrogen for 15 hours or more. It was confirmed that GNA C5 3 batch was reproducibly prepared with an obtained amount (89.4 g, 82.0 g, and 87.1 g), a synthesis yield (119.0%, 109.2%, and 116.0%) and a crude purity (90.3%, 89.4%, and 89.4%) (see the following Table 4). In this case, the molecular weight of the synthesized GNA C5 is 751.04 g/mol (monoisotopic mass: 750.56 g/mol). The molecular weight [M+H]+ measured by a MALDI-TOF apparatus is 751.62 g/mol (see FIG. 8).

TABLE 4

| | Synthetically | | |
|---|---|---|---|
| | obtained amount | Synthesis yield | Purity |
| 1^{st} batch | 89.4 g | 119.0% | 90.3% |
| 2^{nd} batch | 82.0 g | 109.2% | 89.4% |
| 3^{rd} batch | 87.1 g | 116.0% | 89.4% |

** Synthesis scale: 100 mmol

### 8. Purification of GNA C5

GNA C5 crude at a concentration of 0.1 g/mL is added to purified water and completely dissolved. The dissolved GNA C5 crude is filtered with a GF/C filter and a 0.45 μm HVHP membrane filter, and then the filtered product is injected into a column to purify the GNA C5. After purification and lyophilization, GNA C5 3 batch has an obtained amount (49.3 g, 47.2 g, and 48.5 g), a synthesis yield (55.1%, 57.6%, and 55.7%) and a purity (99.1%, 99.0%, and 99.0%) (see the following Table 5).

TABLE 5

| | Amount obtained | | |
|---|---|---|---|
| | from purification | Purification yield | Purity |
| 1^{st} batch | 49.3 g | 55.1% | 99.1% |
| 2^{nd} batch | 47.2 g | 57.6% | 99.0% |
| 3^{rd} batch | 48.5 g | 55.7% | 99.0% |

### Experimental Example 4. Confirmation of Possibility of Mass Synthesis of Ganirelix Acetate

### 1. GNA Convergent Synthesis

GNA C5 (synthesis scale: 34.04 mmol, 39.4 g, 1.54 eq) and HOAt (6.1 g, 1.32 eq) are dissolved in 480 mL of DMF. GNA N5 (28.5 g, 1.0 eq) is slowly added to and dissolved in the reaction solution, and collidine (8.0 g, 1.94 eq) is added thereto. EDC·HCl (11.5 g. 1.76 eq) and DIEA (7.8 g, 1.76 eq) are completely dissolved in 120 mL of DCM. After a DCM solution of dissolved EDC·HCl is slowly added to the reaction solution at 0 to 10° C., the resulting mixture is stirred at 10 to 25° C. for 10 minutes. The reaction solution is drained and collected, the reaction solution is added to 6.0 L of an MTBE:DCM=1:1 (v/v) solution cooled to 10° C. or less, and the resulting mixture is stirred for 30 minutes. To obtain a precipitated solid, the solution was filtered under reduced pressure, the filtered product was washed with 3.0 L of an MTBE:DCM=1:1 (v/v) solution, and then dried under nitrogen for 15 hours or more, and GNA crude was obtained. GNA crude (3 batch) has an obtained amount (52.3 g, 48.6 g, and 51.5 g), a synthesis yield (97.8%, 90.9%, and 96.4%) and a purity (96.8%, 96.2%, and 97.1%) (see the following Table 6). In this case, the molecular weight of the synthesized GNA is 1570.35 g/mol (monoisotopic mass: 1568.84 g/mol). The molecular weight [M+H]+ measured by a MALDI_TOF apparatus is 1570.039 g/mol (see FIG. 9).

TABLE 6

| | Synthetically | | |
|---|---|---|---|
| | obtained amount | Synthesis yield | Purity |
| 1^{st} batch | 52.3 g | 97.8% | 96.8% |
| 2^{nd} batch | 48.6 g | 90.9% | 96.2% |
| 3^{rd} batch | 51.5 g | 96.4% | 97.1% |

** Synthesis scale: 34.04 mmol

### 2. Purification, Salt Substitution and Lyophilization of Crude GNA

Crude GNA is completely dissolved at a concentration of 12.5 mg/mL in 20% acetonitrile (ACN). The dissolved crude GNA is filtered with a GF/C filter and a 0.45 μm HVHP membrane filter, and then the filtered product is injected into a purification column to purify the crude GNA. After purification, salt substitution, and lyophilization, GNA (GNA·2AcOH; a peptide represented by Chemical Formula 11 or 22; 3 batch) has an obtained amount (19.6 g, 26.1 g, and 19.3 g), a yield (39.2%, 58.0%, and 57.6%) and a purity (99.8%, 99.8%, and 99.7%) (see the following Table 7) (Chemical Formula 22: Ac-D-2-Nal-D-Phe(4-Cl)-D-3-Pal-Ser-Tyr-D-hArg(Et)₂-Leu-hArg(Et)₂-Pro-D-Ala-NH₂·2AcOH).

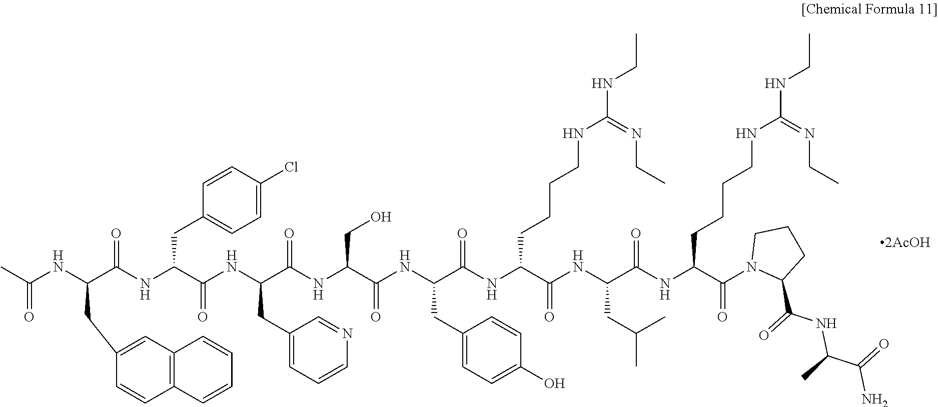 [SMILES: CC/N=C(/NCC)NCCCC[C@H](NC(=O)[C@H](CC(C)C)NC(=O)[C@@H](CCCCN/C(=N\CC)NCC)NC(=O)[C@H](Cc1ccc(O)cc1)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N1CCC[C@H]1C(=O)N[C@H](C)C(N)=O]

TABLE 7

| | Amount obtained | | |
|---|---|---|---|
| | from purification | Purification yield | Purity |
| 1^{st} batch | 19.6 g | 39.2% | 99.8% |
| 2^{nd} batch | 26.1 g | 58.0% | 99.8% |
| 3^{rd} batch | 19.3 g | 57.6% | 99.7% |

### Experimental Example 5. Optimization of GNA N5 Synthesis Process of Example 3-1

### 1. Loading Conditions of First Amino Acid

A step of loading a first amino acid onto a solid support (a 2-chlorotrityl chloride resin, CTC resin) is related to productivity (synthesis yield), and thus affects the synthetic yield of GNA N5. Therefore, as a measure to increase the loading ratio of the first amino acid (Fmoc-Tyr(tBu)-OH), the equivalent of the first amino acid was screened from 1.5 to 5.0 eq. The substitution rate of the CTC resin used in the synthesis is 1.48 mmol/g, and the scale is 10 mmol. The synthesis was carried out at room temperature (25° C.) for 4 hours.

As the amino acid equivalent increases from 1.5 eq to 3.0 to 4.0 eq, the loading ratio increases from 0.75 mmol/g to 0.84 mmol/g, and in the case of 5.0 eq, the loading ratio shows a tendency to decrease to 0.78 mmol/g (see the following Table 8). Therefore, the equivalent of the first amino acid, Fmoc-Tyr (tBu)-OH, was optimized to 3.5 eq.

TABLE 8

| | Amino acid equivalent | | | | | |
|---|---|---|---|---|---|---|
| | 1.5 eq | 2.0 eq | 2.5 eq | 3.0 eq | 4.0 eq | 5.0 eq |
| Loading | 0.75 | 0.76 | 0.79 | 0.84 | 0.84 | 0.78 |
| ratio | | | | | | |
| (mmol/g) | | | | | | |

### 2. Coupling Conditions of Second Amino Acid

Upon coupling of a second amino acid (Fmoc-Ser(tBu)-OH), it was confirmed through HPLC analysis that an unknown soft material was generated at 11.2% at RRT 1.45 (RT 10.8 min). Since this may affect the final GNA N5 and GNA quality (purity), an optimization study for minimizing impurity was carried out.

An attempt was made to minimize the generation of soft materials by adding a base upon coupling of the second amino acid. The effect on purity was observed using several bases. In this case, the types of bases used are 2,4,6-collidine, DIEA, and TEA (basicity: 2,4,6-collidine <DIEA<TEA).

The experimental results are summarized in the following Table 9. When DIEA (2.0 eq) was used, the purity was the highest at 93.6%, and the unknown soft material was also reduced by 0.7% from 11.2%.

TABLE 9

| | | | | | RRT 1.45 |
|---|---|---|---|---|---|
| Base | Amino acid | HOBt | DIC | Purity | impurity |
| No Base | 2.0 eq | 2.2 eq | 2.0 eq | 86.3% | 11.2% |
| Collidine (2.0 eq) | | | | 83.5% | 6.2% |
| DIEA (2.0 eq) | | | | 93.6% | 0.7% |
| TEA (2.0 eq) | | | | 90.2% | 0.6% |

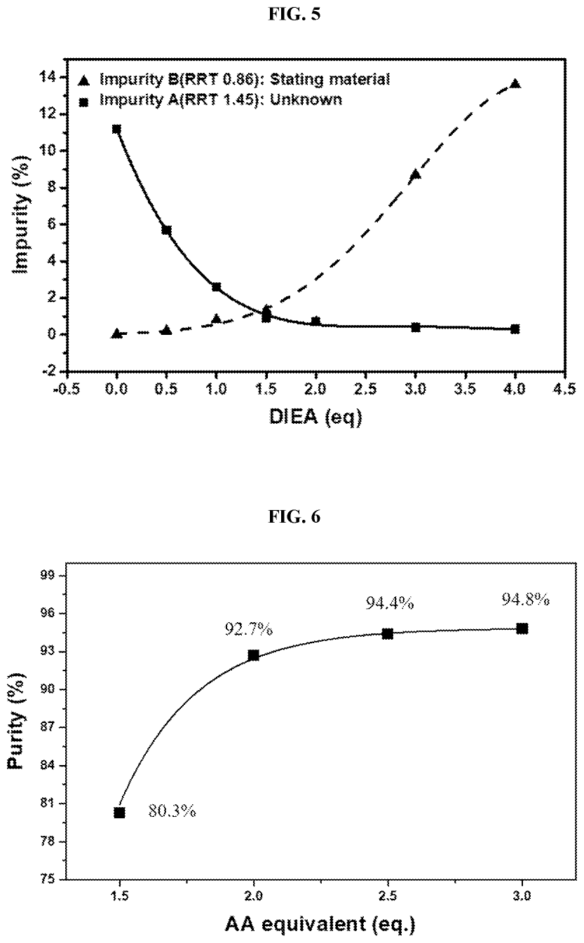

Additionally, optimization research was also conducted on the equivalent of DIEA and the equivalent of the second amino acid. Changes in purity were observed for DIEA in a range of 0 to 4.0 equivalents, and for the second amino acid in a range of 2.0, 2.5, and 3.0 equivalents. FIG. 5 illustrates a soft material change curve with an increasing DIEA equivalent. It was confirmed that as the equivalent of DIEA increases, the amount of unknown soft material decreases (FIG. 5, see square indication line), but due to the increase in unreacted material (starting material, mer) to 13.6%, the purity of the second amino acid is decreased (FIG. 5, see triangle indication line). Therefore, the amount of DIEA used to minimize the unknown soft material was confirmed to be 1.5 equivalents.

Since 1.3% of starting material is present even at the optimum point of DIEA equivalent (1.5 eq), an optimization experiment was performed for the equivalent of the second amino acid in order to minimize the starting material (see the following Table 10). When the second amino acid is present in an amount of 2.0 eq, there is a remaining amount of starting material that is an unreacted product, and when the second amino acid is present in an amount of 3.0 eq, the amount of other impurities increases, resulting in low purity. In contrast, when the second amino acid is present in an amount of 2.5 eq (DIEA: 1.5 eq), there is no remaining amount of starting material and the purity is highest, and therefore the amount used upon the coupling of the second amino acid was selected to be 2.5 eq.

TABLE 10

| | | | | | RRT 1.45 | RRT 0.86 |
|---|---|---|---|---|---|---|
| Amico acid | DIEA | HOBt | DIC | purity | impurity | Impurity (SM) |
| 2.0 eq | 1.5 eq | 2.2 eq | 2.0 eq | 93.2% | 0.9% | 1.3% |
| | | | | 94.2% | 1.0% | 1.7% |
| 2.5 eq | | 2.75 eq | 2.5 eq | 95.9% | 1.2% | — |
| | | | | 95.9% | 1.2% | — |
| 3.0 eq | | 3.3 eq | 3.0 eq | 94.8% | 0.8% | 0.1% |
| | | | | 94.5% | 0.9% | 0.1% |

### 3. Coupling Conditions of Third and Subsequent Amino Acids

It was confirmed that a specific soft material was not produced in the steps of coupling the third to fifth amino acids. However, since there is a possibility that a starting material (deletion of amino acids) may be present due to the shortage of the equivalent of each amino acid, research on optimizing the equivalents of amino acids was conducted. The amount of reaction solvent used in this case was 10 L/mol (volume of reaction solution/reaction scale). FIG. 6 illustrates the results of performing screening to optimize the equivalent of amino acid from the third amino acid coupling step to the acetylation step. A decrease in the purity of GNA N5 was observed when the amount of amino acid was 2.0 eq or less, and there was no difference in GNA N5 purity from 2.5 eq or more. Therefore, the optimized amino acid equivalent was selected to be 2.5 eq.

Additionally, the purity of GNA N5 was evaluated according to the volume of reaction solvent. The amino acid and the activator were used in an amount of 2.5 eq. When the reaction solution was used at 10 L/mol or 20 L/mol, the purity was 96.7% and 96.8%, with no difference.

### 4. Selection of Activator

DIC/HOBt was used as an activator during the synthesis of GNA N5. However, since HOBt, which was used as an additive, is an explosive material and tends not to be used, research for replacing HOBt with Oxyma was conducted. As a result of the experiment, the purity was 95.2% (yield: 128.6%) and 94.6% (yield: 133.8%) when GNA N5 was synthesized using DIC/HOBt and DIC/Oxyma, respectively, with no significant difference. Therefore, Oxyma, which is a safer activator than HOBt, was selected.

### 5. Reaction Temperature Conditions

In order to set standards for the reaction temperature during synthesis, the purity was compared by performing synthesis at 15, 20, 25, and 30° C. (see the following Table 11). Although the impurity pattern was the same according to the temperature, it was confirmed that at 30° C., the 3rd and 5th impurities around the main peak were 0.2% and 2.7%, respectively, which were relatively large compared to other temperatures. Therefore, the synthesis temperature range was set at 20±5° C.

TABLE 11

| | | Impurity | | | | | |
|---|---|---|---|---|---|---|---|
| Temperature | purity | 1 | 2 | 3 | 4 | 5 | 6 |
| 15° C. | 95.3% | 0.4% | 0.3 | 0.06 | 0.3 | 0.2 | 0.4 |
| 10° C. | 94.7% | 0.7% | 0.3 | 0.09 | 0.5 | 0.4 | 0.06 |
| 25° C. | 94.7% | 0.6% | 0.3 | 0.09 | 0.4 | 0.8 | 0.06 |
| 30° C. | 92.7% | 0.5% | 0.3 | 0.2 | 0.3 | 2.7 | 0.1 |

### 6. Global Cleavage and Solidification Process Conditions

Global cleavage and solidification processes are the main processes capable of affecting the purity and yield of GNA N5. There are two protection groups of GNA N5, and optimization research was performed on TFA concentration, temperature, and anti-solvent for global cleavage, which is the step of isolating a peptide from the CTC resin, and the solidification process. In the optimization research on the global cleavage and solidification process, 10 mmol and the amount of cleavage solution used, which is 1.5-fold compared to the volume of the reaction solution (20 L/mol), were used, the reaction was performed for 3 hours, and then purity and yield were compared.

The results of carrying out experiments in which the percentage of TFA was varied from 70% to 95% when global cleavage was performed are summarized in the following Table 12. It was confirmed that when the proportion of TFA was 90% or higher, the purity of GNA N5 was 94.0% or higher, and when the concentration of TFA was 95%, the highest yield (94.4%) was obtained. Therefore, the concentration of TFA during the global cleavage process was selected to be 95%.

TABLE 12

| | TFA | DCM | TIS | H₂O | Total vol. | Yield | Purity |
|---|---|---|---|---|---|---|---|
| 1 | 70% | 25% | 2.5% | 2.5% | 300 mL | 68.1% | 91.9% |
| 2 | 80% | 15% | | | | 86.0% | 92.0% |
| 3 | 90% | 5% | | | | 90.8% | 94.9% |
| 4 | 95% | — | | | | 94.4% | 94.3% |

The solidification reaction was performed by a method of adding the cleavage reaction solution to an anti-solvent. The volume of anti-solvent used was 5-folds compared to that of the cleavage reaction solution. Although there was no significant difference in purity due to temperature change during the solidification process, when solidification was performed using diethyl ether, which is an anti-solvent, at a low temperature (Table 13, Experiment 1: initial −7° C.: A23° C.), the solid state was sticky, making filtration difficult, and therefore it tended to take a long time to obtain GNA N5. In contrast, the higher the initial temperature and solidification temperature, the more solids were formed and the better the filtration (see the following Table 13 and Experiments 3 and 4). When solidification was performed at an initial temperature of 20° C. (the following Table 13 and Experiment 4), the temperature was increased to 38° C. due to instantaneous heat generation. Although the purity was not affected, it is determined that there is a possibility of soft material being generated due to the sudden increase in temperature.

Research was performed to replace diethyl ether, which was used as an anti-solvent in the solidification process and is a highly volatile and flammable material due to its low boiling point, with MTBE which is a relatively safe material. When MTBE was used, it was observed that the solid particles produced during the filtration process after solidification were extremely small in size, and thus passed through the filter as they were (the following Table 13 and Experiment 5; the filtrate through which the solids had passed was refiltered to prevent yield loss). In order to ameliorate the filterability problem, the solidification was carried out by concentrating the cleavage solution. The solidification reaction was performed by a method of adding MTBE to the concentrated cleavage reaction solution. When the solidification was performed by concentrating the cleavage reaction solution to about 40% of the total volume, some of the solids were caught on the filter paper, but some solids still passed through the filter paper as they were (the following Table 13 and Experiment 6; the filtrate through which the solids had passed was refiltered to prevent yield loss). It was confirmed that when the cleavage solution was concentrated to 27%, filtration could be performed well without such problems. Therefore, the degree of concentration was determined to be 30% or less (the following Table 13 and Experiment 7). Furthermore, MTBE was introduced while adjusting the temperature to 20° C. or less. Solid particles became so large that a phenomenon in which particles pass through the filter paper was alleviated. Therefore, MTBE was selected as the anti-solvent during the solidification process.

TABLE 13

| | Temp. | Solvent | Concentration | Yield | Purity | Filtration |
|---|---|---|---|---|---|---|
| 1 | −7° C. → 16° C. | Et₂O | — | 113.7% | 94.3% | Poor |
| | (Δ23° C.) | | | | | |
| 2 | 4° C. → 20° C. | Et₂O | — | 154.9% | 93.8% | Good |
| | (Δ16° C.) | | | | | |
| 3 | 10° C. → 28° C. | Et₂O | — | 131.0% | 96.8% | Very |
| | (Δ18° C.) | | | | | good |
| 4 | 20° C. → 38° C. | Et₂O | — | 105.9% | 97.2% | Very |
| | (Δ18° C.) | | | | | good |
| 5 | 4° C. → 30° C. | MTBE | — | 98.0% | 96.7% | Very |
| | (Δ26° C.) | | | | | poor |
| 6 | −11° C. → 3° C. | MTBE | 300 mL → | 102.2% | 96.4% | Poor |
| | (Δ14° C.) | | 120 mL (40%) | | | |
| 7 | −4° C. → 7° C. | MTBE | 1400 mL → | 99.3% | 95.9% | Good |
| | (Δ11° C.) | | 380 mL (27%) | | | |

### Experimental Example 6. Optimization of GNA C5 Synthesis Process of Example 3-2

### 1. Selection of Activator

The synthesis process of GNA C5 was performed under the conditions shown in the following Table 14. It was confirmed that no specific soft material was produced during the synthesis process. In this case, since HOBt, which was used as an additive, is an explosive material and tends not to be used, research for replacing HOBt with Oxyma was conducted. As a result of the experiment, the purity was 91.5% (yield: 114.7%) and 91.3% (yield: 129.3%) when GNA N5 was synthesized using DIC/HOBt and DIC/Oxyma, respectively, with no significant difference, and therefore Oxyma, which is a safer additive than HOBt, was selected.

TABLE 14

| | | AA | Additive | DIC | Time | Rxn |
|---|---|---|---|---|---|---|
| step | Amino Acid | (eq) | (eq) | (eq) | (h) | Vol. |
| 1 | Fmoc-D-Ala-OH | 2.0 | 2.2 | 2.0 | 3.0 | 10 L/mol |
| 2 | Fmoc-Pro-OH | 2.0 | 2.2 | 2.0 | | |
| 3 | Fmoc-hArg(Et)₂-OH•HCl | 2.0 | 2.2 | 2.0 | | |
| 4 | Fmoc-Leu-OH | 2.0 | 2.2 | 2.0 | | |
| 5 | Fmoc-D-hArg(Et)₂- | 2.0 | 2.2 | 2.0 | | |
| | OH•HCl | | | | | |

### 2. Loading and Coupling Conditions of Amino Acids

Since it may not be possible to adjust the reaction temperature with a reaction liquid volume of 10 L/mol due to the reactor structure during mass production, the reaction was performed by increasing the volume to 20 L/mol, which is a volume that allows the temperature to be easily adjusted (DIC/Oxyma was used as the activator). When 2.0 eq (20 L/mol) of amino acid was synthesized at a reaction solution volume of 20 L/mol, the purity was decreased to 83.9% (yield: 118.7%) due to a 4.4% soft material on HPLC. As a result of analyzing the soft material using Maldi-TOF mass, it was presumed that GNA C5 is a small material having a molecular weight of 226.05, and is due to the deletion of the 3rd amino acid (Fmoc-hArg(Et)₂—OH) or the 5th amino acid (Fmoc-D-hArg(Et)₂—OH). Therefore, the deletion was presumed to have occurred due to an increase in the reaction volume (a decrease in the reaction concentration), thereby increasing the amino acid equivalent to 3.0 eq. As a result, no decrease in purity due to deletion occurred (purity: 91.5%, yield: 114.7%). Therefore, 3.0 eq was selected as the amino acid equivalent suitable for the reaction solution volume of 20 L/mol.

### 3. Reaction Temperature Conditions

In order to set the optimal reaction temperature, the purity was compared by performing synthesis at 15, 20, 25, and 30° C. (see the following Table 15). Although the pattern of the soft material in a temperature range of 15 to 30° C. was the same and did not significantly affect purity, the optimal synthesis temperature range was set at 20±5° C., in the same manner as in that of GNA N5.

TABLE 15

| | | Impurity | | | | | |
|---|---|---|---|---|---|---|---|
| Temperature | purity | 1 | 2 | 3 | 4 | 5 | 6 |
| 15° C. | 89.5% | 0.7% | 1.5% | 0.2% | 0.4% | 1.0% | 1.4% |
| 10° C. | 89.7% | 0.5% | 1.5% | 0.3% | 0.5% | 0.9% | 1.2% |
| 25° C. | 91.0% | 0.6% | 1.5% | 0.2% | 0.5% | 0.8% | 1.0% |
| 30° C. | 89.4% | 0.5% | 1.5% | 0.3% | 0.5% | 0.9% | 1.2% |

### 4. Global Cleavage and Solidification Process Conditions

Global cleavage and solidification processes are the main processes capable of affecting the purity and yield of GNA C5. In the case of GNA C5, since there is no protection group, only GNA C5 may be eliminated from the resin. In order to optimize the global cleavage and solidification process, research was conducted on TFA concentration, temperature, and anti-solvent. In the optimization research on the global cleavage and solidification process, purity and yield were compared after reaction for 3 hours using 10 mmol and the amount of cleavage solution used, which is 1.5-fold compared to the volume of the reaction solution (20 L/mol).

The results of carrying out experiments in which the percentage of TFA was varied from 10% to 80% when global cleavage was performed are summarized in the following Table 16. In the case of GNA C5, since there is no protection group, there is almost no change in purity depending on the concentration of TFA. Although a yield of 107% or more was obtained when the concentration of TFA was 30% or more, GNA C5 was not completely eliminated from the resin at a TFA concentration of 30% or less, and it was confirmed that the yield was shown to be relatively small. Therefore, the optimum concentration of TFA during the global cleavage process was selected to be 40% in consideration of the mass production process.

TABLE 16

| | TFA | DCM | Total vol. | Yield | Purity |
|---|---|---|---|---|---|
| 1 | 80% | 20% | 300 mL | 110.5% | 89.6% |
| 2 | 50% | 50% | | 110.5% | 89.1% |
| 3 | 30% | 70% | | 107.8% | 89.1% |
| 4 | 25% | 75% | | 87.9% | 90.9% |
| 5 | 20% | 80% | | 87.9% | 89.3% |
| 6 | 10% | 90% | | 65.2% | 89.1% |

The solidification reaction was performed by a method of adding the cleavage reaction solution to an anti-solvent. The anti-solvents used in the solidification reaction were diethyl ether and MTBE, and it was confirmed that both solvents could be used for solidification without any problems, and therefore MTBE was selected as it is relatively safe. There was no significant variation in purity and filterability depending on the initial temperature (4.4° C. to 5.5° C.) during the solidification process (see the following Table 17 and Experiments 1, 2, and 3). However, due to its high hygroscopicity, GNA C5 was observed to become a sticky mass over time when the solid obtained after filtration was dried at room temperature. In order to prevent this, such a phenomenon may be avoided when the solid is obtained, and then dried under nitrogen. Furthermore, even though MTBE was used in a volume 7-fold to 10-fold compared to that of the cleavage reaction solution, there were no specificities regarding purity, yield, or filtration.

TABLE 17

| | | | | | | Filtra- |
|---|---|---|---|---|---|---|
| | Temp. | Solvent | Volume | Yield | Purity | tion |
| 1 | 4.4° C.→11.1° C. | Et₂O | 10 folds | 111.8% | 88.4% | Good |
| | (Δ6.7° C.) | | | | | |
| 2 | −0.6° C.→6.8° C. | Et₂O | 10 folds | 102.5% | 90.3% | Good |
| | (Δ7.4° C.) | | | | | |
| 3 | −5.5° C.→2.4° C. | Et₂O | 10 folds | 107.8% | 89.6% | Good |
| | (Δ7.9° C.) | | | | | |
| 4 | −5.3° C.→3.2° C. | MTBE | 10 folds | 105.2% | 89.8% | Good |
| | (Δ8.5° C.) | | | | | |
| 5 | −5.0° C.→7.0° C. | MTBE | 7 folds | 121.3% | 91.1% | Good |
| | (Δ12.0° C.) | | | | | |

### Experimental Example 7. Optimization of GNA Convergent Synthesis Process of Example 3-3

### 1. Selection and Conditions of Activator

In order to minimize the soft material produced during GNA convergent synthesis, screening research on activators was carried out. The synthesis scale was 1.14 mmol, and the results are summarized in the following Table 18. The soft material of RRT1.04 is located just behind the main peak and is quite difficult to remove during purification, and therefore the present inventors attempted to select an activator capable of producing the least amount of this soft material. In experiments using EDC·HCl/DEPBT, TOTU, and EDC·HCl/HOAt among various activators, a soft material (RRT1.04) was produced at 2.0% or less, and thus this was selected as a candidate material. Although TOTU was the lowest at 1.2% of the soft materials in GNA crude, it was excluded because it is not dissolved well in crude and is expensive. DEPBT was also excluded because it is expensive and is difficult to supply. Therefore, EDC·HCl/HOAt was selected as the most suitable activator because it is relatively inexpensive and easy to supply, and produces less soft material (RRT1.04).

TABLE 18

| | | | | Impurity |
|---|---|---|---|---|
| | Activator | Yield | Purity | RRT 1.04 |
| 1 | EDC•HCl/HOBt | 101.1% (1.81 g) | 94.9% | 3.7% |
| 2 | EDC•HCl/Oxyma | 95.0% (1.70 g) | 95.5% | 2.8% |
| 3 | EDC•HCl/DEPBT | 97.8% (1.75 g) | 95.3% | 1.8% |
| 4 | HATU | 96.6% (1.73 g) | 96.4% | 2.2% |
| 5 | HCTU | 97.2% (1.74 g) | 92.7% | 5.3% |
| 6 | TBTU | 112.8% (2.02 g) | 86.0% | 9.8% |
| 7 | TCTU | 110.1% (1.97 g) | 93.6% | 5.1% |
| 8 | TOTU | 92.7% (1.66 g) | 97.1% | 1.2% |
| 9 | TPTU | 95.0% (1.70 g) | 74.8% | 18.0% |
| 10 | EDC•HCl/HOAt | 92.7% (1.66 g) | 95.7% | 1.9% |
| 11 | HBTU | 106.1% (1.90 g) | 85.7% | 10.5% |

Optimization research on EDC·HCl equivalents (0.88 eq, 1.32 eq, 1.76 eq, and 2.20 eq) was performed. The synthesis scale was 0.57 mmol. As the equivalent of EDC·HCl was increased from 0.88 eq to 1.76 eq, the purity of GNA crude was increased from 64.9% to 97.4%, and the remaining amount of starting material GNA N5 was decreased from 33.1% to 0.1%. When the EDC·HCl equivalent was 2.20 eq, the purity was decreased slightly to 96.9%, but the remaining amount of GNA was 0.03% (see the following Table 19). Based on the above results, the EDC·HCl equivalent was optimized to 1.76 eq.

TABLE 19

| | | | | GNA N5 |
|---|---|---|---|---|
| | | | | remaining |
| | EDC•HCl (eq.) | Yield | Purity | amount |
| 1 | 0.88 | 108.9% (0.86 g) | 64.9% | 33.1% |
| 2 | 1.32 | 107.6% (0.85 g) | 96.5% | 0.7% |
| 3 | 1.76 | 103.8% (0.82 g) | 97.4% | 0.1% |
| 4 | 2.20 | 105.1% (0.83 g) | 96.9% | 0.03% |

Research for optimizing the equivalent of HOAt was carried out. The synthesis scale was 1.14 mmol. As HOAt was increased from 0.44 eq to 1.32 eq, the soft material (RRT 1.04) was decreased from 2.4% to 1.8%, and when HOAt was increased from 1.32 eq to 1.76 eq, the degree of decrease was 1.7%, which was not significant (see the following Table 20). Therefore, HOAt was optimized to 1.32 eq.

TABLE 20

| | | | | Soft material |
|---|---|---|---|---|
| | HOAt (eq.) | Yield | Purity | RRT 1.04 |
| 1 | 0.44 | 106.4% (1.67 g) | 96.4% | 2.4% |
| 2 | 0.88 | 104.5% (1.64 g) | 96.8% | 2.0% |
| 3 | 1.32 | 108.9% (1.71 g) | 96.8% | 1.8% |
| 4 | 1.76 | 106.4% (1.67 g) | 96.9% | 1.7% |

### 2. Optimization by DOE

In the convergent synthesis method, GNA C5, EDC·HCl, and collidine equivalent are set as process variables through the design of experiments (DOE) method to carry out optimization research for interactions between reagents and intermediates and each process variable. Further, the present inventors intended to derive a process operation range by deriving a design space. Experiments were sequentially performed by designing the equivalent change of each variable (including the center point) and the experimental sequence using DOE specialized software (Fusion pro). The change ranges in each equivalent of GNA C5, EDC·HCl, and collidine are 1.28 to 1.92 eq, 0.88 to 2.64 eq, and 0.88 to 2.64 eq. The synthesis scale was 0.57 mmol, and the experimental sequence and results (purity) are shown in the following Table 21.

TABLE 21

| Run no. | GNA C5 (eq.) | EDC•HCl(eq.) | Collidine (eq.) | Purity |
|---|---|---|---|---|
| 1 | 1.92 | 1.76 | 1.76 | 94.5% |
| 2 | 1.67 | 2.64 | 1.76 | 94.3% |
| 3 | 1.28 | 2.64 | 2.64 | 92.4% |
| 4 | 1.28 | 0.88 | 2.64 | 80.0% |
| 5 | 1.92 | 2.64 | 2.64 | 94.6% |
| 6 | 1.92 | 0.88 | 0.88 | 21.7% |
| 7 | 1.92 | 0.88 | 2.64 | 66.1% |
| 8 | 1.67 | 1.76 | 1.76 | 95.2% |
| 9 | 1.67 | 1.76 | 0.88 | 94.9% |
| 10 | 1.28 | 2.64 | 0.88 | 92.7% |
| 11 | 1.67 | 1.76 | 2.64 | 95.5% |
| 12 | 1.28 | 1.76 | 1.76 | 92.8% |
| 13 | 1.92 | 2.64 | 0.88 | 95.6% |
| 14 | 1.28 | 0.88 | 0.88 | 53.7% |
| 15 | 1.67 | 0.88 | 1.76 | 64.7% |

There was no interaction between variables in the experiment. Among the three variables, EDC was fixed at 1.76 eq, GNA purity was set to 94% or higher, and a design space was derived for the operating range of two variables (GNA C5, collidine) capable of achieving the same. In the design space, the operating range of GNA C5 is 1.54 eq to 1.78 eq (center point: 1.65 eq), and the operating range of collidine is 1.72 eq to 2.25 eq (center point: 1.99). A target purity of 94% may be achieved when any equivalent weight is used within this range. To prove this, a verification experiment was carried out to see if the purity was 94% or higher when an experiment was performed at the equivalents of GNA C5 and collidine at each vertex and center point in the design space. The synthesis scale was 1.14 mmol, and the results are shown as in the following Table 22. Similar to the experimental results, the purity was higher than the target purity (94%) at all points. Therefore, it was verified that the equivalent was appropriately set as the optimal operating range for GNA C5 and collidine.

TABLE 22

| Point | GNA C5 (eq) | EDC•HCl(eq.) | Collidine (eq.) | purity |
|---|---|---|---|---|
| A | 1.54 | 1.76 | 1.72 | 95.5% |
| B | 1.78 | 1.76 | 1.72 | 95.9% |
| C | 1.54 | 1.76 | 2.25 | 95.7% |
| D | 1.78 | 1.76 | 2.25 | 96.0% |
| T | 1.65 | 1.76 | 1.99 | 96.1% |

The scope of the present invention is represented by the following claims, and it should be interpreted that the meaning and scope of the claims and all the changes or modified forms derived from the equivalent concepts thereof fall within the scope of the present invention.

1. A method for manufacturing ganirelix, the method comprising:
obtaining a peptide intermediate A represented by the following Chemical Formula 19;
obtaining a peptide intermediate B represented by the following Chemical Formula 21; and
obtaining ganirelix represented by the following Chemical Formula 13 through a convergent synthesis of the peptide intermediate A and the peptide intermediate B:
Ac-D-2-Nal-D-Phe(4-Cl)-D-3-Pal-Ser-Tyr-OH [Chemical Formula 19]
H₂N-D-hArg(Et)₂-Leu-hArg(Et)₂-Pro-D-Ala-NH₂ [Chemical Formula 21]
Ac-D-2-Nal-D-Phe(4-Cl)-D-3-Pal-Ser-Tyr-D-hArg(Et)₂-Leu-Arg(Et)₂-Pro-D-Ala-NH₂. [Chemical Formula 13]

2. The method of claim 1, wherein the obtaining of the peptide intermediate A comprises:
obtaining a peptide represented by the following Chemical Formula 18; and
removing a protecting group and a resin from the peptide represented by Chemical Formula 18:
Ac-D-2-Nal-D-Phe(4-Cl)-D-3-Pal-Ser(R¹)-Tyr(R¹)—O-Resin [Chemical Formula 18]
wherein R¹ is hydrogen or a hydroxyl group-protecting group.

3. The method of claim 2, wherein the resin is a 2-chlorotrityl resin, a trityl resin, a 4-methyltrityl resin, a 4-methoxytrityl resin, or a 4-methylbenzhydrylamine (MBHA) resin.

4. The method of claim 2, wherein the removing of the protecting group and the resin comprises:
reacting the peptide represented by Chemical Formula 18 with a mixed solution comprising a combination of items selected from the group consisting of trifluoroacetic acid (TFA), triisopropylsilane (TIS), dichloromethane (DCM), ethylenedioxy diethanethiol (DODT), dimethylsulfide (DMS) and ammonium iodide (NH₄I); and
mixing diethyl ether (Et₂O), tert-Butyl methyl ether (MTBE), or a combination thereof with a reaction solution thereof to get a resulting mixture and solidifying the resulting mixture.

5. The method of claim 1, wherein the obtaining of the peptide intermediate A is performed in the presence of an activator comprising 2,4,6-collidine, 1-hydroxybenzotriazole (HOBt), ethyl(hydroxyimino)cyanoacetate (Oxyma), N,N′-diisopropylcarbodiimide (DIC), 1-ethyl-3-(3-dimethylaminopropyl) carbodiimide) hydrochloride (EDC·HCl), 1-hydroxy-7-azabenzotriazole (HOAt), 3-(diethoxyphosphoryloxy)-1,2,3-benzotriazin-4(3H)-one (DEPBT), bis(dimethylamino)methylene]-1H-1,2,3-triazolo[4,5-b]pyridinium-3-oxide hexafluorophosphate (HATU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethylaminium tetrafluoroborate (TBTU), 1-[bis(dimethylamino)methylen]-5-chlorobenzotriazolium 3-oxide tetrafluoroborate (TCTU), N-[[(1-cyano-2-ethoxy-2-oxoethylidene)amino]oxy](dimethylamino)methylene]-N-methyl-methanaminium tetrafluoroborate (TOTU), 2-(2-pyridon-1-yl)-1,1,3,3-tetramethyluronium tetrafluoroborate (TPTU), N,N,N′,N′-tetramethyl-O-(1H-benzotriazol-1-yl) uronium hexafluorophosphate (HBTU), or a combination thereof.

6. The method of claim 1, wherein the obtaining of the peptide intermediate A comprises coupling Ser as a second amino acid in the presence of the following basic reagent:
2,4,6-collidine, pyridine, imidazole, pyrrolidine, cyclohexylamine, morpholine, piperidine, 4-methoxypyridine, 2-chloropyridine, 4-dimethylaminopyridine, aniline, 4-methoxyaniline, 4-phenylenediamine, ethylamine, diethylamine, triethylamine, N,N-diisopropylethylamine (DIEA), 1,8-diazabicyclo[5.4.0]undec-7-ene (DBU), or a combination thereof.

7. The method of claim 6, wherein the obtaining of the peptide intermediate A comprises coupling Ser as a second amino acid in the presence of the basic reagent in an amount of 0.5 eq to 4 eq.

8. The method of claim 1, wherein the obtaining of the peptide intermediate B comprises:
obtaining a peptide represented by the following Chemical Formula 20;
removing a resin from the obtained peptide represented by Chemical Formula 20; and
obtaining intermediate B by purifying the peptide from which the resin has been removed:
H₂N-D-hArg(Et)₂-Leu-hArg(Et)₂-Pro-D-Ala-NH-Resin. [Chemical Formula 20]

9. The method of claim 8, wherein the resin is a 2-chlorotrityl resin, a trityl resin, a 4-methyltrityl resin, a 4-methoxytrityl resin, or a 4-methylbenzhydrylamine (MBHA) resin.

10. The method of claim 8, wherein the removing of the resin comprises:
reacting the peptide represented by Chemical Formula 20 with a mixed solution comprising a combination of items selected from the group consisting of trifluoroacetic acid (TFA), triisopropylsilane (TIS), dichloromethane (DCM), ethylenedioxy diethanethiol (DODT), dimethylsulfide (DMS) and ammonium iodide (NH₄I); and
mixing diethyl ether (Et₂O), tert-Butyl methyl ether (MTBE), or a combination thereof with a reaction solution thereof and solidifying the resulting mixture.

11. The method of claim 1, wherein the obtaining of intermediate B is performed in the presence of an activator comprising 2,4,6-collidine, 1-hydroxybenzotriazole (HOBt), ethyl(hydroxyimino)cyanoacetate (Oxyma), N,N′-diisopropylcarbodiimide (DIC), 1-ethyl-3-(3-dimethylaminopropyl) carbodiimide) hydrochloride (EDC·HCl), 1-hydroxy-7-azabenzotriazole (HOAt), 3-(diethoxyphosphoryloxy)-1,2,3-benzotriazin-4(3H)-one (DEPBT), bis(dimethylamino)methylene]-1H-1,2,3-triazolo[4,5-b]pyridinium-3-oxide hexafluorophosphate (HATU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethylaminium tetrafluoroborate (TBTU), 1-[bis(dimethylamino)methylen]-5-chlorobenzotriazolium 3-oxide tetrafluoroborate (TCTU), N-[[[(1-cyano-2-ethoxy-2-oxoethylidene)amino]oxy](dimethylamino)methylene]-N-methyl-methanaminium tetrafluoroborate (TOTU), 2-(2-pyridon-1-yl)-1,1,3,3-tetramethyluronium tetrafluoroborate (TPTU), N,N,N′,N′-tetramethyl-O-(1H-benzotriazol-1-yl) uronium hexafluorophosphate (HBTU), or a combination thereof.

12. The method of claim 1, wherein the obtaining of ganirelix is performed in the presence of a coupling reagent comprising 2,4,6-collidine, 1-hydroxybenzotriazole (HOBt), ethyl(hydroxyimino)cyanoacetate (Oxyma), N,N′-diisopropylcarbodiimide (DIC), 1-ethyl-3-(3-dimethylaminopropyl) carbodiimide) hydrochloride (EDC·HCl), 1-hydroxy-7-azabenzotriazole (HOAt), 3-(diethoxyphosphoryloxy)-1,2,3-benzotriazin-4(3H)-one (DEPBT), bis(dimethylamino)methylene]-1H-1,2,3-triazolo[4,5-b]pyridinium-3-oxide hexafluorophosphate (HATU), O-(1H-6-chlorobenzotriazole-1-yl)-1,1,3,3-tetramethyluronium hexafluorophosphate (HCTU), 2-(1H-benzotriazole-1-yl)-1,1,3,3-tetramethylaminium tetrafluoroborate (TBTU), 1-[bis(dimethylamino)methylen]-5-chlorobenzotriazolium 3-oxide tetrafluoroborate (TCTU), N-[[(1-cyano-2-ethoxy-2-oxoethylidene)amino]oxy](dimethylamino)methylene]-N-methyl-methanaminium tetrafluoroborate (TOTU), 2-(2-pyridon-1-yl)-1,1,3,3-tetramethyluronium tetrafluoroborate (TPTU), N,N,N′,N′-tetramethyl-O-(1H-benzotriazol-1-yl) uronium hexafluorophosphate (HBTU), or a combination thereof.

13. The method of claim 1, wherein the obtaining of ganirelix is performed in the presence of 0.5 eq to 4 eq of EDC·HCl.

14. The method of claim 1, wherein the obtaining of ganirelix is performed in the presence of 0.2 eq to 3 eq of HOAt.

15. The method of claim 12, wherein the obtaining of ganirelix comprises subjecting intermediate A and intermediate B to convergent synthesis in the presence of 1.5 eq to 2.5 eq of the coupling reagent.

16. The method of claim 12, wherein the obtaining of ganirelix comprises:
performing a convergent synthesis reaction on intermediate A and intermediate B in the presence of the coupling reagent; and
mixing dichloromethane (DCM), tert-Butyl methyl ether (MTBE), or a combination thereof with a reaction solution thereof and solidifying the resulting mixture.

17. The method of claim 16, wherein, in the solidifying of the resulting mixture, DCM and MTBE are mixed at a volume ratio (v/v) of 1:2 to 2:1 in the reaction solution and the resulting mixture is solidified.

18. A method for manufacturing ganirelix acetate, the method comprising: obtaining a peptide intermediate A represented by the following Chemical Formula 19;
obtaining a peptide intermediate B represented by the following Chemical Formula 21;
obtaining ganirelix represented by the following Chemical Formula 13 through the convergent synthesis of intermediate A and intermediate B; and
obtaining ganirelix acetate represented by the following Chemical Formula 22 by purifying the obtained ganirelix and substituting the purified ganirelix with an acetate:
Ac-D-2-Nal-D-Phe(4-Cl)-D-3-Pal-Ser-Tyr-OH [Chemical Formula 19]
H₂N-D-hArg(Et)₂-Leu-hArg(Et)₂-Pro-D-Ala-NH₂ [Chemical Formula 21]
Ac-D-2-Nal-D-Phe(4-Cl)-D-3-Pal-Ser-Tyr-D-hArg(Et)₂-Leu-Arg(Et)₂-Pro-D-Ala-NH₂ [Chemical Formula 13]
Ac-D-2-Nal-D-Phe(4-Cl)-D-3-Pal-Ser-Tyr-D-hArg(Et)₂-Leu-hArg(Et)₂-Pro-D-Ala-NH₂·2AcOH. [Chemical Formula 22]
